(19) United States
(12) Patent Application Publication
(10) Pub. No.: US 2016/0169801 A1
(43) Pub. Date: Jun. 16, 2016

(54) Target Characterization Based on Persistent Collocation of Multiple Specks of Light in Time Series Imagery

(71) Applicant: THE BOARD OF TRUSTEES OF THE LELAND STANFORD JUNIOR UNIVERSITY, Palo Alto, CA (US)

(72) Inventors: Anita Rogacs, Redwood City, CA (US); Lewis A. Marshall, Pleasanton, CA (US); Juan G. Santiago, Stanford, CA (US)

(73) Assignee: The Board of Trustees of the Leland Stanford Junior University, Palo Alto, CA (US)

(21) Appl. No.: 14/891,310

(22) PCT Filed: May 9, 2014
(86) PCT No.: PCT/US14/37599

§ 371 Date: Nov. 13, 2015

Related U.S. Application Data
(60) Provisional application No. 61/822,340, filed on May 11, 2013.

(51) Int. Cl. G01N 21/64; G01N 33/58; G01N 33/53; G06T 7/20; C12Q 1/68

(52) U.S. Cl. CPC G01N 21/6428; G06T 7/20; C12Q 1/6825; G01N 33/53; G01N 33/582; G01N 2021/6439; G01N 2021/6441; G06T 2207/30204

(57) ABSTRACT

Techniques for characterizing targets include obtaining multiple time series of images. Each image represents light measured in an interrogation area under conditions that cause only one optical marker type of at least two optical marker types to emit or scatter light. Each different time series indicates light measured from a different single optical marker type. The at least two optical marker types are configured to collocate with a single target type. The techniques include determining a path of a speck of light from an individual optical marker of a first optical marker type. The techniques also include determining whether the path corresponds to the target type based on persistence of collocation of a speck of light from each of the other optical marker types. The collocation can be based on maximum correlation in a portion of contemporaneous images. The persistence can be long compared to random separation times.

### CROSS-REFERENCE TO RELATED APPLICATIONS

This application claims benefit of Provisional application 61/822,340, filed May 11, 2013, the entire contents of which are hereby incorporated by reference as if fully set forth herein, under 35 U.S.C. §119(e).

### BACKGROUND OF THE INVENTION

Accompanying the growing number of particle, cell, and bead-based assay applications, particle and cell analysis methods have become more rigorous and sophisticated. The two most frequently cited techniques are likely flow cytometry (FCM) and laser-scanning cytometry (LSC). Flow cytometry is a workhorse technology used routinely in immunology, pathology, and hemotology. FCM performs a single high content multiparametric measurement for thousands of particles in minutes or less. (Here “particles” denote living and fixed cells and/or micron-scale fluorescent beads such as Luminex beads.) Microparticles are particles with a largest dimension less than about 1000 microns (1 micron, also called a micrometer, m, 1 m=10⁻⁶ meters) and include nanoparticles with a largest dimension in a range from about 1 nanometer (nm, 1 nm=10⁻⁹ meters) to about 1000 nm.

The core of a standard cytometer is a flow chamber where a particle-laden stream is hydrodynamically focused with the aid of sheath flow into a small interrogation region through which particles traverse, often one at a time. Lasers illuminate particles in the interrogation region. Forward light scatter is approximately correlated to the size of the particle and side scatter contains particle granularity information. Increasing need for multiplexing via polychromatic excitation and emission has pushed the frontiers of flow cytometry, and enabled, for example, implementation of instruments that measure up to 19 parameters (17 fluorescent colors and 2 physical parameters).

LSC was originally designed to provide an imaging complement to traditional FCM and allow morphological analysis of adherent cells. LSC scans stationary particles (typically cells) adhered to a surface and its implementation has evolved to include analyses of cell proliferation, tissue architecture, and immunophenotyping using precious samples. The cells are fixed onto slides and scanned with multiple lasers. This can be repeated over time for studies of enzyme kinetics and other time-resolved processes. The slide can be removed from the instrument to change staining or otherwise modify the cells; and then placed back on the instrument for re-analysis. Each cell can be relocated and all of the data points can be correlated for multivariate analysis.

There is a range of other multicolor particle image counting and analysis systems. For example, recently the field has seen development of imaging versions of FCM systems wherein point detectors are replaced by high speed imaging to analyze particle morphology in more detail and using up to 12 wavelengths (e.g., the Amnis Imagestream system). These systems employ time delay integration (TDI) wherein the particle-specific detection region of the camera is panned electronically to track the cells in the flow stream. These systems can produce up to 12 simultaneous realizations of a darkfield, two brightfield images, and nine fluorescence images of each particle in suspension.

The bead-target-particle hybrid assay, developed by Mirkin and coworkers, relies on two-component oligonucleotide-modified gold nanoparticles (NPs) and single-component oligonucleotide-modified magnetic microparticles (MMPs). Both the MMPs and the NPs contain probes complimentary to a target sequence. In addition, NPs contain access probes complimentary to bar-code sequences, which hold a unique ID for the target of interest. MMPs and then NPs are added to a solution containing a mixture of bar-code sequences and target DNA. After separation of the doublets from the unbound beads and free barcode sequences using a magnetic field, the doublets are washed and the bound barcode sequences are released and hybridized on a microarray for detection.

More recently, Leslie at al. described a method of DNA detection involving direct monitoring of the binding events between two oligo-functionalized magnetic beads and a target sequence. The assay combines DNA and superparamagnetic beads in a rotating magnetic field and produces multiparticle aggregates detectable with the naked eye. Quantification of the dark area resulting from aggregated beads is correlated with the concentration of target DNA. Due to the nature of aggregates, multiplexing using this assay is limited.

### SUMMARY OF THE INVENTION

There remains a need for better methods that will allow rapid detection of targets (such as molecules, organelles and cells) with high sensitivity even in multiplex assays in which different targets are identified in the same sample during the same process. Techniques are provided for simple and computational efficient characterization of targets based on multiple imaging conditions.

As used hereinafter, a target is anything that can be carried by a fluid in a channel, and includes large molecules, beads, cells, and portions of cells (e.g., organelles), among other structures. Examples of large molecules include chains of amino acids (polypeptides and proteins) and chains of nucleic acid bases (oligonucleotides, ribonucleic acid [RNA], and deoxyribonucleic acid [DNA]). A target type is a member of a family of targets that share one or more physical or chemical properties that define the type. Thus, an oligonucleotide type is a member of a family of oligonucleotide molecules that share a particular nucleotide sequence that defines the type. Similarly, a polypeptide type is a member of a family of polypeptide molecules that share a particular amino acid sequence that defines the type; a cell type is a member of a family of cells that express a set of proteins or oligonucleotides (either internally or as receptors on its surface or some combination) that defines the type.

Light refers to electromagnetic radiation in an optical range of wavelengths that includes the visible, infrared and ultraviolet, with wavelengths in a range from about 200 nm to about 2000 nm. As used herein, optical markers are objects that emit or scatter light at a known optical wavelength or in a known optical wavelength band (also called a spectral band or spectral channel), either spontaneously or upon stimulation with a known stimulus, such as heat or light of the same wavelength or band (for reflectance) or different wavelength or band (for fluorescence). As used herein, the term interrogation conditions refers to the type of excitation (including no excitation) and the light produced as a result of such excitation. An optical marker collocates with a target when the optical marker binds to, is incorporated within, or is produced by the target. An optical marker type is a member of a family of optical markers with the same interrogation conditions that collocate with the same one or more target types.

As used herein, an “interrogation region” is a portion of a fluidic channel where light emitted or scattered from an optical marker is monitored, and, in some embodiments, where the optical marker is excited. Interrogation regions are often transparent. As used herein, a detectable amount of light emitted or scattered (reflected or fluorescently returned) from an optical marker, e.g., when the optical marker is properly excited, is called a speck of light, or simply a speck.

In a first set of embodiments, a method includes providing a supply of at least two optical marker types that scatter or emit light under a corresponding number of different conditions. The at least two optical marker types are configured to collocate with a single target type. A fluid that includes the supply is introduced into an interrogation region of a channel. The interrogation region is serially imaged under the corresponding number of different conditions to produce multiple time series of images. Each time series of images detects light from a single optical marker type of the at least two optical marker types. The method includes determining, in a first time series, a path of a speck of light from an individual optical marker of a first optical marker type. The method also includes determining whether the path corresponds to the target type based on persistence of collocation of the speck of light from the individual optical marker of the first optical marker type with a speck of light from each of other optical marker types different from the first optical marker type.

In some embodiments of the first set, persistence of collocation of the speck of light from each of the other optical marker types is based on persistence of a correlation measure above a correlation threshold. In some of these embodiments, the correlation measure is a maximum correlation among a plurality of correlations within a collocation area between corresponding portions of contemporaneous images of two of the multiple time series.

In some embodiments of the first set, the corresponding number of different conditions includes: scattering or emitting light of the same optical wavelength in response to different excitations sources; or scattering or emitting light of different optical wavelength in response to the same or different excitations sources; or emitting light of different optical wavelengths without excitation.

In some embodiments of the first set, providing the supply further comprises providing a supply of a multiplexed optical marker type that scatters or emits light under a corresponding different multiplexed condition and is configured to collocate at a single different target type with at least one other optical marker type. In these embodiments, serially imaging the interrogation region includes serially imaging the interrogation region under conditions that cause the multiplexed optical marker to emit light, to produce a multiplexed time series of images. In these embodiments, the method includes determining a path of a speck of light from the multiplexed optical marker type; and, determining whether the path corresponds to the multiplexed target type based on persistence of collocation of a speck of light from the at least one other optical marker type.

In a second set of embodiments, a method includes obtaining multiple time series of images, each image representing light measured in an interrogation area of a fluid under conditions that cause only one optical marker type of at least two optical marker types to emit or scatter light. Each different time series of images indicates light measured from a different single optical marker type of the at least two optical marker types. The at least two optical marker types are configured to collocate with a single target type. The method also includes determining on a processor, in the time series of images, a path of a speck of light from an individual optical marker of a first optical marker type of the at least two optical marker types. The method still further includes determining on a processor whether the path corresponds to the target type based on persistence of collocation of the speck of light from the individual optical marker of the first optical marker type with a speck of light from each of the other optical marker types different from the first optical marker type.

In some embodiments of the second set, persistence of collocation of the speck of light from each of the other optical marker types is based on persistence of a correlation measure above a correlation threshold. In some of these embodiments, the correlation measure is a maximum correlation among a plurality of correlations within a collocation area between corresponding portions of contemporaneous images of two of the multiple time series.

In some embodiments of the second set, the collocation area is based on an expected size for the target type and values for zero or more parameters that include expected sizes of the at least two optical marker types, expected positions of the at least two optical marker types on the target type, and expected number of pixels in an optical system over which is spread the speck of light from the at least two optical marker types.

In some embodiments of the second set, determining whether the path corresponds to the target type includes determining whether the persistence time is greater than a time for an optical marker to move randomly out of the collocation area, such as by fluid diffusion or by Brownian particle motion.

In a third set of embodiments, a non-transitory computer-readable medium carries one or more sequences of instructions, wherein execution of the one or more sequences of instructions by one or more processors causes an apparatus to obtain multiple time series of images. Each image represents light measured in an interrogation area of a fluid under conditions that cause only one optical marker type of at least two optical marker types to emit or scatter light. Each different time series of images indicates light measured from a different single optical marker type of the at least two optical marker types. The at least two optical marker types are configured to collocate with a single target type. The instructions further cause the apparatus to determine, in the time series of images, a path of a speck of light from an individual optical marker of a first optical marker type of the at least two optical marker types. The instructions still further cause the apparatus to determine whether the path corresponds to the target type based on persistence of collocation of the speck of light from the individual optical marker of the first optical marker type with a speck of light from each of other optical marker types different from the first optical marker type.

In a fourth set of embodiments, a system includes at least one processor and at least one memory including one or more sequences of instructions. The at least one memory and the one or more sequences of instructions are configured to, with the at least one processor, cause an apparatus to obtain multiple time series of images. Each image represents light measured in an interrogation area of a fluid under conditions that cause only one optical marker type of at least two optical marker types to emit or scatter light. Each different time series of images indicates light measured from a different single optical marker type of the at least two optical marker types. The at least two optical marker types are configured to collocate with a single target type. The instructions further cause the apparatus to determine, in the time series of images, a path of a speck of light from an individual optical marker of a first optical marker type of the at least two optical marker types. The instructions still further cause the apparatus to determine whether the path corresponds to the target type based on persistence of collocation of the speck of light from the individual optical marker of the first optical marker type with a speck of light from each of other optical marker types different from the first optical marker type.

In a fifth set of embodiments, a kit includes a supply, for each target type, of at least two optical marker types that scatter or emit light under a corresponding number of different conditions and are all configured to collocate only with the target type. The kit also includes a computer-readable medium carrying one or more sequences of instructions, wherein execution of the one or more sequences of instructions by one or more processors causes the one or more processor to obtain multiple time series of images. Each image represents light measured in an interrogation area of a fluid under conditions that cause only one optical marker type of at least two optical marker types to emit or scatter light. Each different time series of images indicates light measured from a different single optical marker type of the at least two optical marker types. The at least two optical marker types are configured to collocate with a single target type. The instructions further cause the apparatus to determine, in the time series of images, a path of a speck of light from an individual optical marker of a first optical marker type of the at least two optical marker types. The instructions still further cause the apparatus to determine whether the path corresponds to the target type based on persistence of collocation of the speck of light from the individual optical marker of the first optical marker type with a speck of light from each of other optical marker types different from the first optical marker type

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A is a block diagram that illustrates an example quadruple view of a sample in a channel;

FIG. 1B is a block diagram that illustrates example capture of light from a single optical marker at multiple pixels of an imaging system;

FIG. 1C is a graph that illustrates an example for deducing location of an optical marker from light received at multiple pixels of FIG. 1B, according to an embodiment;

FIG. 2A is a block diagram that illustrates an example target collocated with a pair of optical markers, according to an embodiment;

FIG. 2B is a block diagram that illustrates an example different target collocated with a different pair of optical markers, according to an embodiment;

FIG. 3A is a block diagram that illustrates an example fluid flow with a mixture of non-targets, targets and optical markers, according to an embodiment;

FIG. 3B is a block diagram that illustrates an example track of a target collocated with a pair of optical markers, according to an embodiment;

FIG. 3C is a block diagram that illustrates an example track of a non-target collocated with only one optical marker of a pair of optical markers, according to an embodiment;

FIG. 4A is a flow diagram that illustrates an experimental method to characterize targets, according to an embodiment;

FIG. 4B is a flow diagram that illustrates a computation method to perform a step of the method of FIG. 4A, according to an embodiment;

FIG. 5 is a block diagram that illustrates an example alignment of two views from a quadruple view optical imager, according to an embodiment;

FIG. 6A through FIG. 6C are block diagrams that illustrate an example experiment with beads as optical markers and a DNA type as a target type, according to an embodiment;

FIG. 7 is a graph that illustrates example dependence of performance on signal to noise ratio (SNR), according to an embodiment;

FIG. 8 is a graph that illustrates example dependence of performance on various adjustable parameters, according to various embodiments;

FIG. 9 is a graph that indicates persistent correlation of specks in two spectral bands from an experiment as described in FIG. 6A through FIG. 6C, according to an embodiment;

FIG. 10 is a block diagram that illustrates a computer system upon which an embodiment of the invention may be implemented; and

FIG. 11 illustrates a chip set upon which an embodiment of the invention may be implemented.

### DETAILED DESCRIPTION

A method, system and kit are described for characterizing targets, including microparticles, based on persistent collocation of multiple specks of light in time series imagery. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of fluorescent beads as optical markers and a DNA molecule type as a target type. However, the invention is not limited to this context. In other embodiments the target types are other DNA types or other oligonucleotide types or protein types or cell types or organelle types or other nanostructures or microstructures. In some embodiments, the target types are not even microparticles. In other embodiments, the optical markers are light emitting diodes, or quantum dots, or fluorophores or naturally occurring fluorescent proteins, either attached to molecules that bind to the targets or ingested by target cells, or produced by target cells, either naturally or after genetic modifications to the cells, or built into target manufactured structures or microstructures.

### 1. Overview

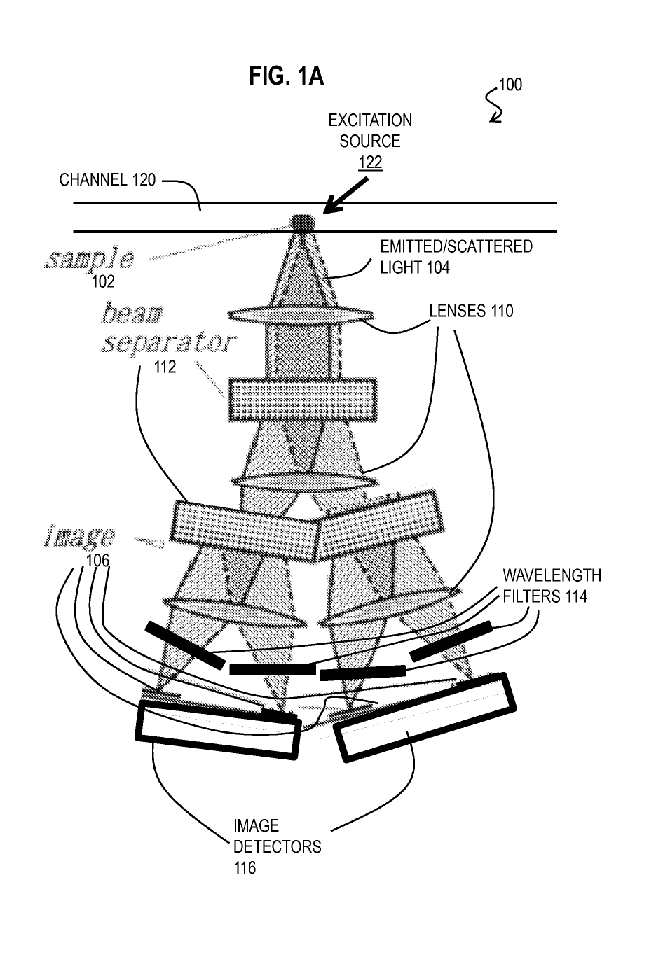

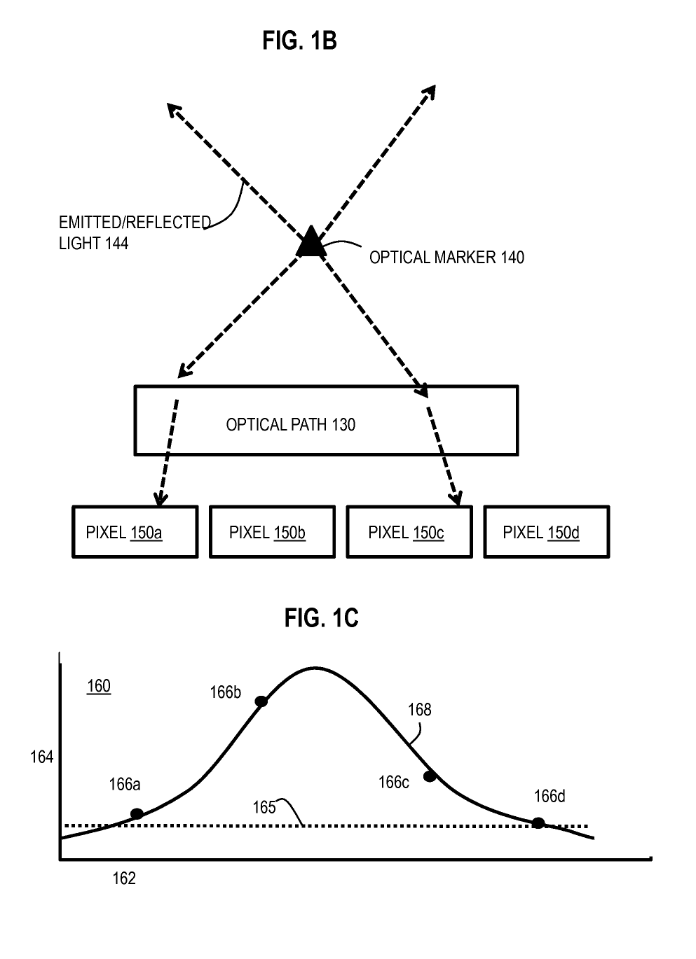

Various embodiments use existing equipment in new ways to characterize target in a fluid or fluid flow. FIG. 1A is a block diagram that illustrates an example quadruple view of a sample 102 in a channel 120. The quadruple view imaging system 100 uses lenses 110 and beam separators (also called beam splitters) 112 to divide the light 104 emitted or scattered from a sample 102 into four images 106 captured by one or more image detectors 116, such as charge coupled device (CCD) arrays. Filters 114 are employed to pass only light in a particular spectral band. Thus images in up to four different spectral bands can be obtained from the same sample 102 at the same time. Other devices can be used to obtain other numbers of spectral bands simultaneously from one sample 102. Many such dual view and quadruple view devices are known in the art, and any can be used in various embodiments. For example is some embodiments, a Micro-Imager, from Photometric of Tucson, Ariz., is used as a quadruple view imager. In some embodiments, a prism or grating is used instead of one or more beam splitters 112 to send different wavelengths into different directions. In some such embodiments, one or more filters 114 are omitted.

In some embodiments, the sample 102 emits light in response to an excitation source 122, such as a heat source or wideband light source or narrowband laser. When a laser or light source is used, a fluorescent response, which emits light at a different wavelength from a wavelength or band of the excitation source 122, is easily distinguished from stray light from the source 122. In some embodiments, the source 122 includes one or more optical components to direct the excitation energy onto the sample 102 in the channel 120.

FIG. 1B is a block diagram that illustrates example capture of light from a single optical marker at multiple pixels of an imaging system. An optical marker 140 emits or scatters light 144, e.g., in response to excitation from source 122. An optical path 130, such as one of the paths that produces one of the images depicted in FIG. 1A, directs some of the emitted light into one or more pixels, such as pixels 150a, 150b, 150c, 150d, of a detector array to generate a detected speck of light. Even though the optical marker 140 is smaller than a pixel, in some embodiments, the emitted or scattered light may fall on multiple pixels, as depicted in FIG. 1B. By fitting a peak function to the measured intensities at the different pixels, a subpixel position can be attributed to the optical marker 140. FIG. 1C is a graph 160 that illustrates an example for deducing location of an optical marker from a speck of light that spans multiple pixels of FIG. 1B, according to an embodiment. The horizontal axis 162 indicates position in one direction along the image space, the vertical axis 164 indicates intensity in the spectral band impinging on the pixels. Points 166a, 166b, 166c and 166d indicate an intensity measurements made above noise level 165 at each pixel and ascribed to the midpoint of the pixel. Trace 168 is a functional form (e.g., Gaussian) fit through the points 166a through 166d. The location of the peak in trace 168, indicates the probable subpixel location of the optical marker 140. Many such peak functional forms are known and used in the art; and, any can be used in various embodiments described herein.

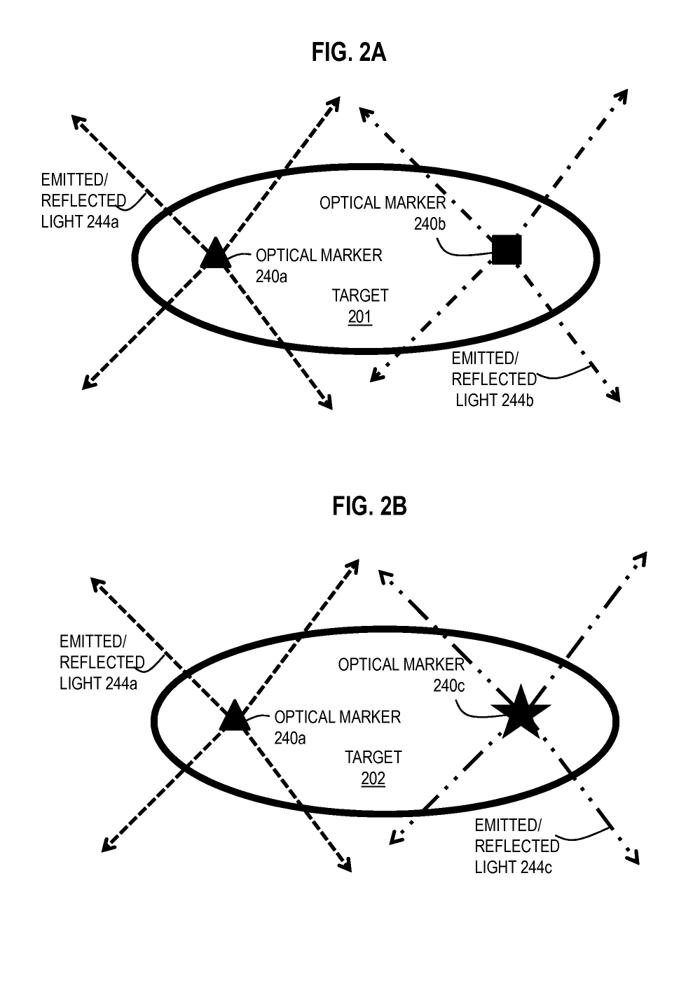

FIG. 2A is a block diagram that illustrates an example target of a first target type 201 collocated with a pair of optical markers of corresponding different optical marker types, 240a and 240b, respectively, according to an embodiment. Optical marker type 240a scatters or emits light 244a at one wavelength or narrow wavelength band, either spontaneously or in response to excitation; and, optical marker type 240b emits light 244b at a distinguishable wavelength or narrow wavelength band, either spontaneously or in response to excitation. The light 244b is distinguishable from the light 244a either by being in a different wavelength or wavelength band, having a different intensity, or by being excited by a different source, e.g., a laser at a different wavelength than a laser that excites optical marker 240a.

Many optical scatterers are known in the art and any can be used in various embodiments. Fluorescent dyes that can be attached to molecules for co-locating with a target type include streptavidin-phycoerythrin (SA-PE, Life Technologies), a 300 KDa fluorophore, Alexa Fluor® 350, Alexa Fluor® 647, Oregon Green®, Alexa Fluor® 405, Alexa Fluor® 680, Fluorescein (FITC), Alexa Fluor® 488, Alexa Fluor® 750, Cy®3, Alexa Fluor® 532, Pacific Blue™, Pacific Orange™, Alexa Fluor® 546, Coumarin Tetramethylrhodamine (TRITC), Alexa Fluor® 555, BODIPY® FL, Texas Red®, Alexa Fluor® 568, Pacific Green™ Cy®5, and Alexa Fluor® 594. Example DNA stains include DAPI, SYTOX® Green, SYTO® 9, TO-PRO®-3, and Propidium Iodide. Example quantum dots include Qdot® 525, Qdot® 565, Qdot® 605, Qdot® 655, Qdot® 705, and Qdot® 800. Fluorescent proteins include R-Phycoerythrin (R-PE) and Allophycocyanin (APC). Genetically expressed fluorescent proteins include CFP, GFP (emGFP), and RFP (tagRFP).

In some embodiments, one or both optical markers are fluorescent beads, which vary in a size from about 20 nm to about 10 μm. The choice of a particular size bead is based on a tradeoff between sensitivity and time needed to detect a binding event. The diffusivity of small beads is greater, so the target-bead reactions occur more quickly, which is advantageous in some embodiments. The binding event can also be detected in a shorter amount of time as the randomly correlated small beads will separate very quickly. However, detecting the light from 20 nm beads is more difficult than detecting the light from 20 μm beads. So in some embodiments it is advantageous to select a larger bead and cope with the longer reaction times and longer times to separate randomly collocated beads. In some of these embodiments, several different bead types emit at the same wavelength or narrow wavelength band, but at different intensities, e.g., beads available from Luminex™ Corporation of Austin Tex. are available that fluoresce at ten different levels of intensity.

In some embodiments using beads, one or more molecules that bind to one or more molecules or structures of the target are adhered to the bead to render it suitable for a particular function or purpose, using any methods known in the art. For example, nucleic acids can be covalently attached to micro-spheres with any of several methods. Carboxyl and amino groups are the most common reactive groups for attaching ligands to surfaces. These groups are very stable over time, and their chemistries have been widely explored. Several reactive groups can be incorporated on the bead surface for covalent coupling, including: —COOH, Carboxylic acid; —RNH2, Primary aliphatic amine; —ArNH2, Aromatic amine; —ArCH2Cl, Chloromethyl (vinyl benzyl chloride); —CONH2, Amide; —CONHNH2, Hydrazide; —CHO, Aldehyde; —OH, Hydroxyl; —SH, Thiol; and —COC—, Epoxy. Attaching an amino group to the 5′ or 3′ end of an oligonucleotide or a PCR primer is straightforward and inexpensive. See, for example, Integrated DNA Technologies, “Strategies for Attaching Oligonucleotides to Solid Supports,” published by Integrated DNA Technologies, Inc. (Coralville, Iowa, 2010: 19 pp).

In various embodiments, the target type is an oligonucleotide type, a RNA type, a DNA type, a polypeptide type, a protein type, a cell type, or a manufactured structure type.

FIG. 2B is a block diagram that illustrates an example different target of a different target type 202 collocated with a different pair of optical markers of a different pair of optical marker types 240a and 240c, according to an embodiment. In the illustrated embodiment, the same optical marker type 240a is used as one of the two optical marker types collocated with target type 202, but a different optical marker type is used for the second optical marker type. Optical marker type 240b emits light 244b at a different wavelength or different narrow wavelength band, or intensity, either spontaneously or in response to the same or different excitation. Thus the combination or optical marker types 240a and 240c is unique for target type 202 and distinguishable from the pair of optical marker types 240a and 240b used for target type 201. In other embodiments, two different optical markers are used for a different target type. In some embodiments, the different optical marker, e.g., 240c emits at the same wavelength as 240b, but at a different distinguishable intensity level, for example, using Luminex beads available at ten different distinguishable intensity levels.

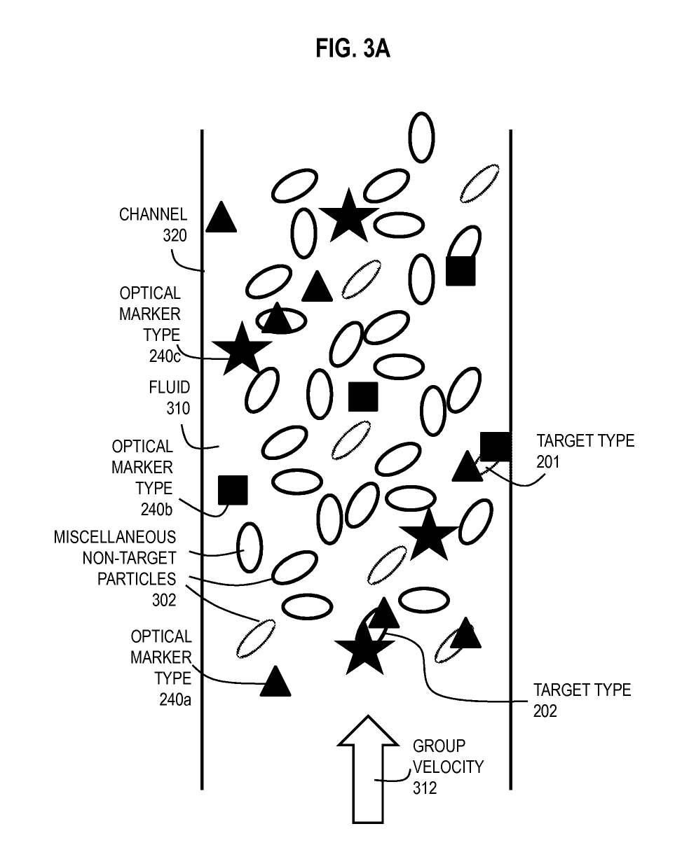

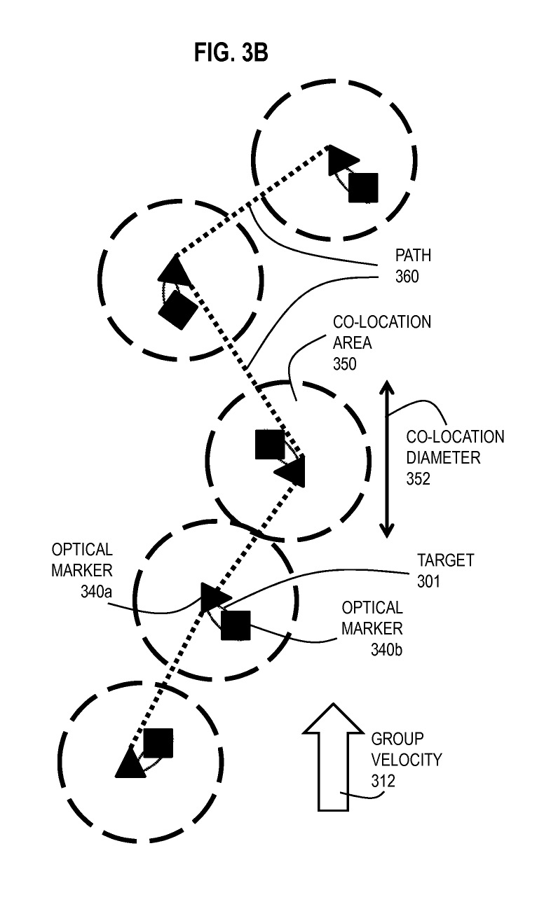

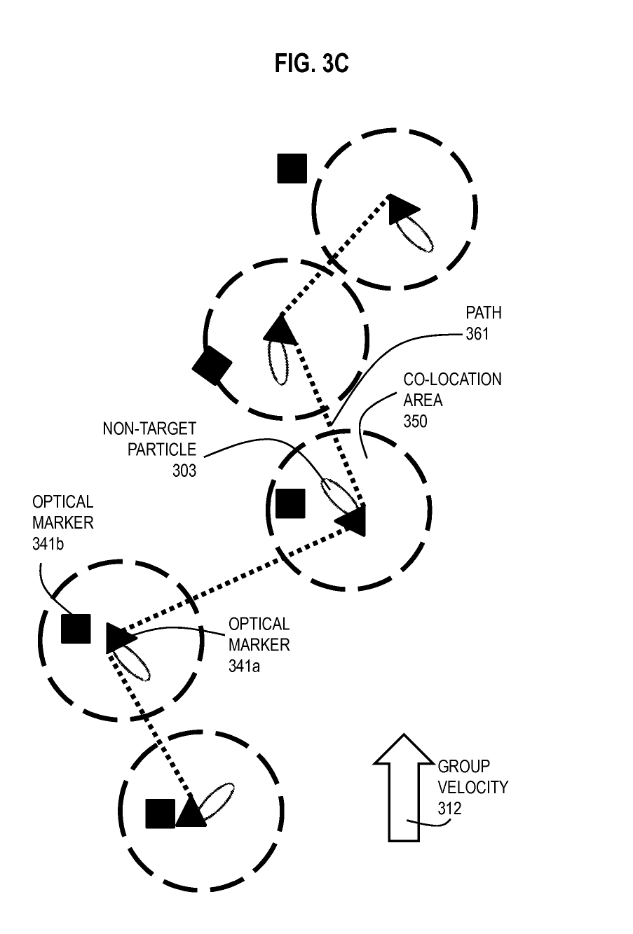

FIG. 3A is a block diagram that illustrates an example fluid flow with a mixture of miscellaneous non-target particles 302, target types 201, 202 and optical marker types 240a, 240b, 240c, according to an embodiment. The fluid 310 flows with a speed and direction that matches a group velocity 312 that carries the optical marker typess 240a, 240b and 240c along with the fluid 310. In some embodiments, the fluid is not adverting but instead, the particles are propelled through the fluid at group velocity 312, e.g. using an electric fields (e.g., in electrophoresis) or magnetic fields (e.g., in magnetophoresis). A particle of target type 201 is shown after binding to optical markers of the optical marker types 240a and 240b. Similarly, a particle of different target type 202 is shown after binding to optical markers of the optical marker types 240a and 240c. According to various embodiments, in an optical apparatus like that depicted in FIG. 1A, the particles of the two types are distinguished from each other and the free optical markers, and optical markers bound to other target types, by the persistence of the collocation of specks of light from the corresponding pair of optical marker types, as the various particles move with or through the fluid 310.

FIG. 3B is a block diagram that illustrates an example path 360 of an individual target 301 of a first target type 201, which is collocated with a pair of optical markers 340a and 340b, of a first pair of different optical marker types, 240a and 240b, respectively, according to an embodiment. In various images of a time series of images in an interrogation area of an apparatus such as depicted in FIG. 1A, a particle moving in or with fluid will generally follow the group velocity 312 with some random motion superposed (e.g., from some combination of diffusivity and Brownian motion). FIG. 3B depicts the same particle 301 at successive different locations as occupied in different successive images. The successive locations of a speck of light from the optical marker 340a (e.g., as ascertained using the curve fitting of FIG. 1B), which is bound to the target 301, are connected by segments of path 360. Surrounding each location is a circular collocation area 350 with diameter 352. In other embodiments, the collocation area is not circular; for example, the collocation area is square or rectangular or takes the shape of some other polyhedron. For particles of the first target type 201 which have bound to both optical marker types 240a and 240b, a speck of light from an optical marker 340a of type 240b will always be observed within the collocation area 350. In contrast, FIG. 3C shows that a particle of a different type, that does not bind to both optical marker types 240 and 240b, will eventually show a diffusion of the speck of light from unbound optical marker of type 240b until the speck lies outside of the collocation area.

FIG. 3C is a block diagram that illustrates an example path 361 of a non-target particle 303 collocated with only one optical marker 341a of only one optical marker type 240a of a pair of different optical marker types 240a and 240b, according to an embodiment. While the particle 303 binds to and stays with the first optical marker 341a and its path 361, the second optical marker 341b, of the second optical marker type 240b, eventually diffuses out of the collocation area. Thus collocation of specks of light from two optical markers in FIG. 3C, e.g., specks within collocation area 350 centered on the speck from optical marker 240a, does not persist as long as collocation of specks of light from the two optical markers in FIG. 3B. Thus non-target particle 303 in FIG. 3C is distinguished from target particle 301 of target type 201 in FIG. 3B.

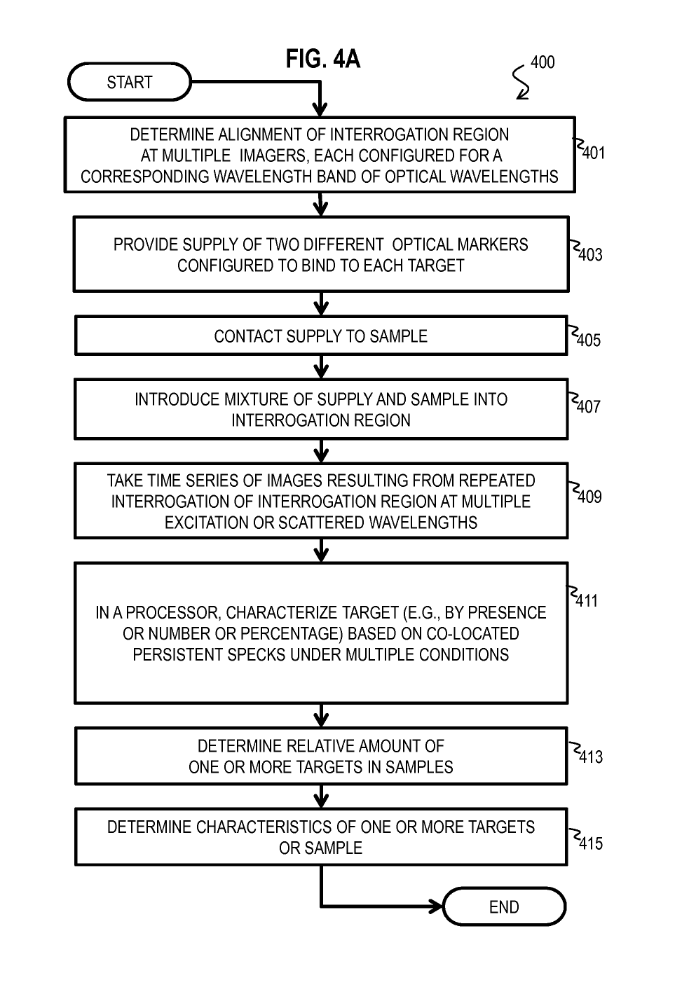

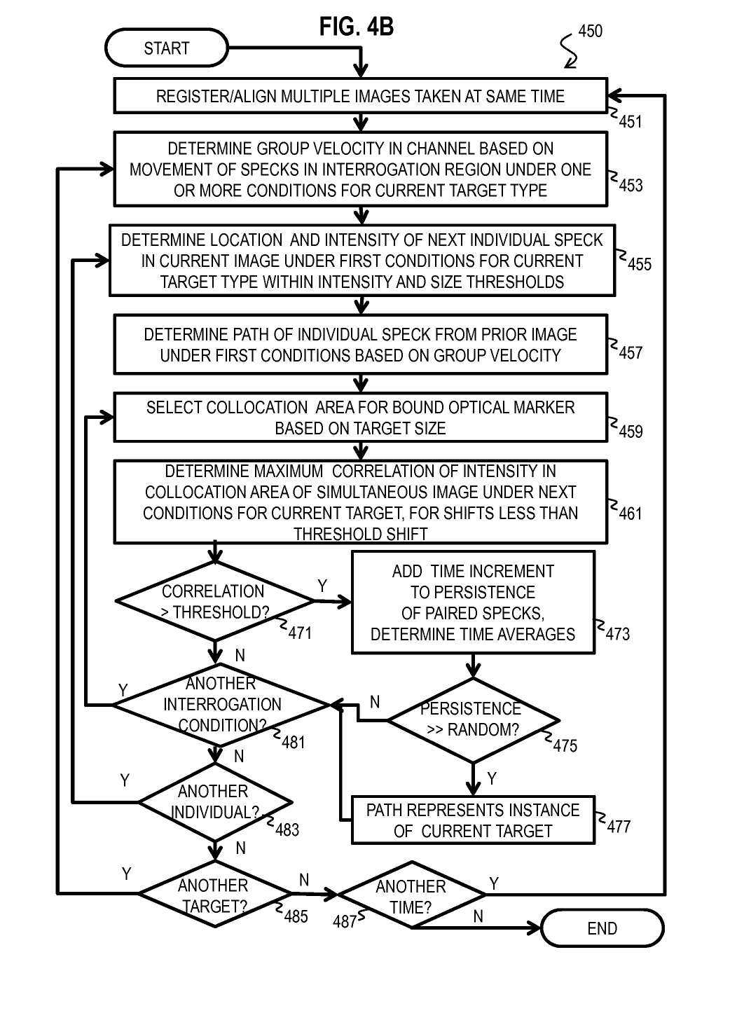

FIG. 4A is a flow diagram that illustrates an experimental method 400 to characterize targets, according to an embodiment. Although steps are depicted in FIG. 4A, and in subsequent flowchart FIG. 4B, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. For example, the steps are presented below as if done in real time as each image in a time series is obtained. In other embodiments, the data is first collected and then the steps are done in a different order as post processing after data collection. In other embodiments, some steps are done during data collection and other steps are done as post processing.

In step 401, the view of an interrogation region of a fluidic channel in each of two or more optical paths of a multiple view optical apparatus, such as a Quad view device, are aligned. Each view is configured to be used different conditions (e.g., spectral bands of excitation or detection or both). For example, a bright field pattern is inserted into the interrogation region and detected in each of the two or more optical paths. The optical components or computed pixel positions or both are adjusted so that the patterns overlap within about one pixel. This process is called image registration. The parameters of this transformation are saved. In an example embodiment, a rigid body transformation (constant translation and rotation) is used to register the multiple views.

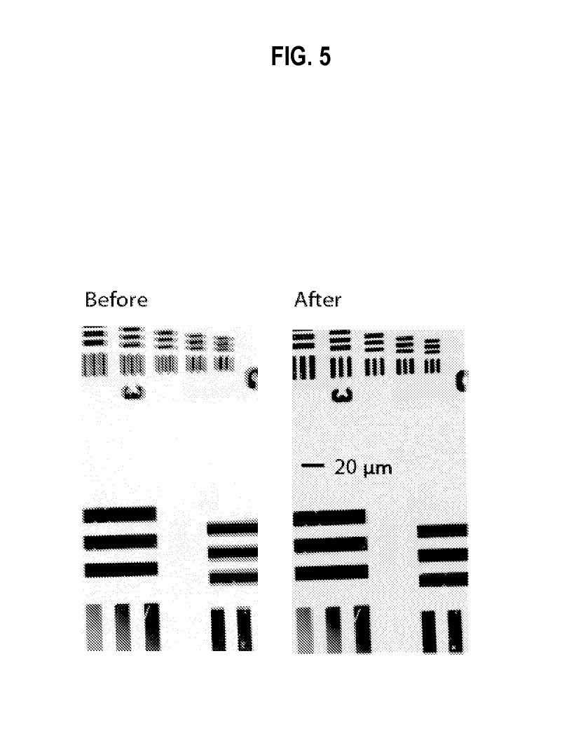

FIG. 5 is a block diagram that illustrates an example alignment of two views from a quadruple view optical imager, according to an embodiment. In this example, a bright field image of an alignment pattern (Negative 1951 wheel pattern resolution test target from Thorlabs, R3L1S4N) is used. Initial misaligned images (left) and aligned images (right) of two spectral channels recorded with a quad-view imager (Micro-Imager from Photometrics of Tucson, Ariz.), are shown here in dual-view mode. The images are plotted using function imshowpair in MATLAB® from The MathWorks®, Inc. of Natick, Mass., which displays the differences between two images. Alpha blending is used to overlay the two spectral channel images before (left) and after (right) image registration. Alpha blending is the process of combining a translucent foreground color with a background color, which produces a new blended color. These test images were taken with a 20× objective with a numerical aperture of 0.5. The function imregister of MATLAB® is used in an iterative process that involves a pair of images, an image-similarity metric, an optimizer, and a transformation type. The metric defines the image similarity for evaluating the accuracy of the registration. The optimizer defines the methodology for minimizing or maximizing the similarity metric. The MATLAB® function imconfig was used to generate the optimizer and metric assuming multimodal image capture, as the brightness range in the two spectral channels are different. The transformation type used for the image alignment is specified as “rigid”, which consists of translation (Δi, Δj), and rotation (α). The maximum number of iterations was set to 1000.

The region of interest (ROI) coordinates and the transformation matrix generated in this alignment phase are then used to translate and rotate an image of specks of light from one or more optical markers in a second spectral channel to achieve spatial registration with an image of specks of light from one or more different optical markers in a first spectral channel. Using images of simulated specks patterns; it has been verified that this alignment procedure results in registration with sub-pixel accuracy. Note, the image alignment and registration process produces artifacts at the perimeter of the images, which are eliminated by trimming about 5 pixels from the image edges. It is advantageous to perform this registration process only periodically as a part of the instrumentation calibration procedure, to save time and expense.

Returning to FIG. 4A, in step 403, a supply is provided that includes optical markers of two or more optical marker types configured to bind to each of one or more target types, such that each different target type binds to an unique observable combination of optical marker types. For example one or more cells of a target cell type are to be genetically engineered to express a fluorescent protein type as a first optical marker type when another particular set of one or more genes of the target type are also expressed, and a second optical marker type is configured to collocate with the cell same type. In various embodiments, the second optical marker type is caused to collocate by also genetically engineering the same cell type to express a different second fluorescent protein type as a second optical marker type when a different particular set of one or more genes of the target type are also expressed. In such embodiments, the supply of optical maker types includes DNA representing genes that express the one or more fluorescent markers. In other or different embodiments, the first or second optical marker type is a dye type or bead type configured to bind to a receptor of the target cell type or a stain type to bind to a particular structure type, such as the nucleus of a cell type. Thus, step 403 includes providing a supply of at least two optical marker types that emit or scatter light at a corresponding number of different conditions and are all configured to collocate with a single target type.

In step 405, the supply of optical marker types for one or more target types are brought in contact with a sample that potentially includes or produces one or more target types. For example, in some embodiments, step 405 includes genetically altering one or more cells, or producing a buffering fluid into which the markers or genetically engineered cells are introduced. In some embodiments, a sample includes powder from grinding, slices from a microtome or chemicals from lysating a portion of a solid structure or tissue, either dry or in a buffering solution, or a fluid extracted from a subject or including particles from a subject. In various embodiments, contacting includes dropping the supply with optical marker types into the sample, or the sample into the supply, or both into a buffering solution.

In some embodiments, step 405 includes collecting a sample from a subject. The subject may be any device or organism, including bacteria, plants and animals (including humans and other mammals), which potentially has or produces one or more target types. In some embodiments, a sample is a biological sample that may be extracted, untreated, treated, diluted, or concentrated from a subject. Any cell type or tissue typemay be used in various embodiments. DNA testing can be performed from any bodily fluid that includes genomic DNA, e.g., blood, tissue biopsies, hair follicles or skin. For prenatal subjects, fetal nucleic acid samples can be obtained from maternal blood, amniocytes or chorionic villi. Suitably, the biological sample is selected from any part of a subject's body, including, but not limited to hair, skin, nails, tissues or bodily fluids such as saliva, blood, plasma, and serum.

Thus, in some embodiments, the target type is a cell type that expresses a first particular molecule and a second particular molecule. In some of these embodiments, the cell type has been genetically engineered so that a first fluorescent protein that serves as a first one of the at least two optical marker types is expressed when the first particular molecule is expressed. In some of these embodiments, the cell type has been genetically engineered further so that a different second fluorescent protein that serves as a different second one of the at least two optical marker types is expressed when the second particular molecule is expressed. In other of these embodiments, the second particular molecule is a receptor on a membrane of the cell type; and, a second fluorescent labeled antibody that serves as a different second one of the at least two optical marker types binds to the receptor.

In some embodiments, the first particular molecule is a receptor on a membrane of the cell type; and, a first fluorescent antibody that serves as a first one of the at least two optical markers binds to the receptor. In some of these embodiments, the second particular molecule is a different receptor on the membrane of the cell type; and, a second fluorescent antibody that serves as a different second one of the at least two optical markers binds to the different receptor.

In some embodiments, the target type is any DNA molecule with a particular unique sequence of bases. Thus, in some of these embodiments, a first fluorescent labeled probe that binds to a first portion of the particular unique sequence serves as a first marker type of the at least two optical marker types; and, a second fluorescent labeled probe that binds to a different second portion of the particular unique sequence serves as a different second marker type of the at least two optical marker types. In some of these embodiments, at least one of the first labeled probe and the second labeled probe comprises a fluorescent bead attached to a molecule that binds to a corresponding portion of the first portion of the particular unique sequence or the second portion of the particular unique sequence. As used here, a labeled probe includes a label and a portion configured to bind to a specific target molecule or bind to a specific portion of a target molecule.

In some embodiments, one or more of the labeled probes includes as a label a Luminex bead with different calibrated levels of emission intensity at a given optical wavelength of emission. Thus, in these embodiments, when interrogated, the fluorescent bead emits in a corresponding optical wavelength of the corresponding number of different optical wavelengths at a particular intensity level selected from a plurality of different intensity levels.

In step 407 the mixture of optical marker supply and sample is introduced into an interrogation region of a fluidic channel, such as a microfluidic channel. Any method may be used to introduce or propel the fluid through the interrogation region, including hydrostatic pressure, air pressure, pumps and electrophoresis and magnetophoresis. In some embodiments, the fluid undergoes no net motion; and the targets or beads or some combination is propelled, e.g., using electrophoresis or magnetophoresis or some combination. Thus, in some embodiments, step 407 includes introducing a fluid that includes the supply into an interrogation region of a channel. In some embodiments, the fluid and or particles are not propelled, but are simply introduced into the channel and do not undergo net motion. In such embodiments, the same scene is interrogated repeatedly, or the interrogation region is moved along the fluidic channel either by moving the optical system or sliding a chip that contains the fluid channel past a stationary optical system, or some combination.

In step 409, a time series of images are taken of the interrogation region in each of several conditions that distinguish the different optical marker types, e.g., looking at specks of light in multiple spectral bands, by repeatedly interrogating optical marker types in the interrogation region. In some embodiments, interrogation includes stimulating the optical markers, by heating or excitation at one or more optical wavelengths that elicit a fluorescent response from the optical marker types collocated with one or more target types of interest. Light emitted in the interrogation region is directed by one or more optical components onto two or more image detectors, configured to record light in corresponding one or more different spectral bands. Thus, step 409 includes serially imaging the interrogation region under conditions that cause the at least two optical markers to emit or scatter light, to produce a time series of images in each of the corresponding number of different conditions. It is advantageous to distinguish the two optical markers to collect images of their emissions in different image detectors or different portions of the same image detector. Thus the spectral bands of the detectors can be broad and even overlap, so long as the specks of light from different optical markers are not detected in the same image detector or portion thereof.

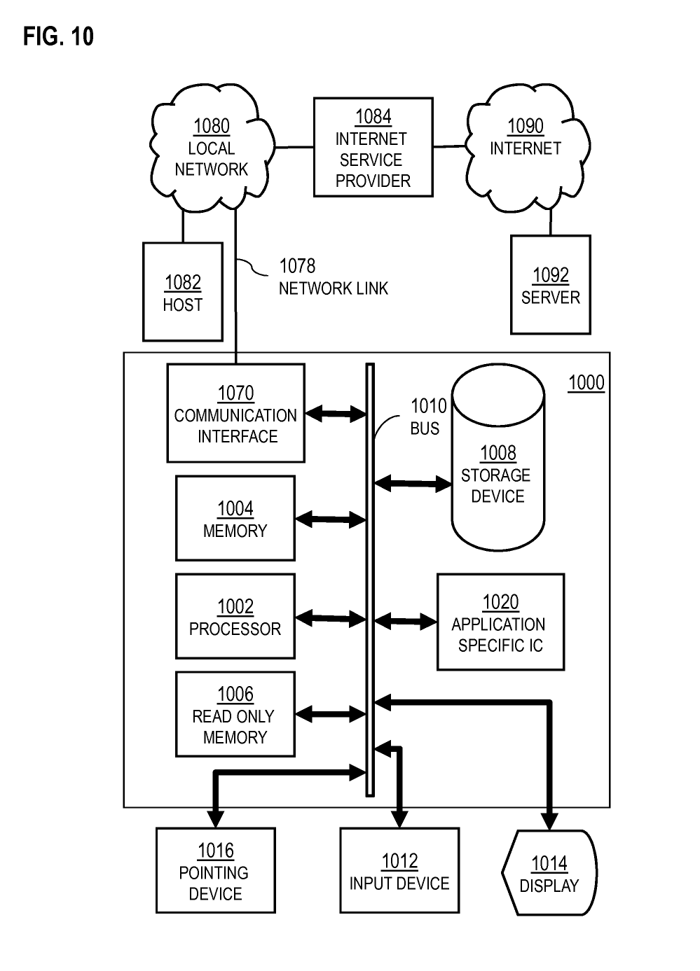

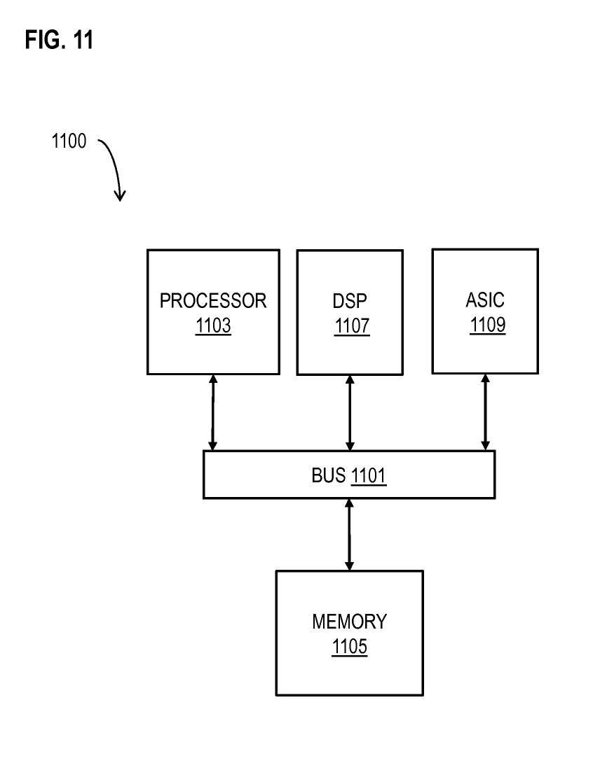

In step 411, the one or more target types are characterized by using a processor, such as the computer system of FIG. 10 or chip set of FIG. 11, to determine the temporal persistence of collocated scatterers in two or more spectral bands. A particular embodiment of step 411 is described in the subsequent flow chart of FIG. 4B, described in more detail below. In general terms, step 411 includes determining paths of individual specks of light from a single optical marker type, and determining the persistence of a speck of light from a second different optical marker type. (and, in some embodiments, from other different optical marker types) in the vicinity of the individual speck from the first optical marker type. The vicinity is defined in terms of a collocation area, such as collocation area 350 depicted in FIG. 3B and FIG. 3C. Thus, step 411 includes determining, in a first time series of the plurality of time series of images, a path of a speck of light from an individual optical marker of a first optical marker type of the at least two optical marker types. Step 411 also includes determining whether the path corresponds to the target type based on persistence of collocation of a speck of light from each of other optical marker types of the at least two optical marker types, wherein the other optical marker types are different from the first optical marker type.

In some embodiments using beads with different intensity levels for a given optical wavelength of emission, determining whether the track corresponds to the target type further comprises determining whether the track corresponds to the target type based on persistence of collocation and intensity of a speck of light from each of the other optical marker types.

In some embodiments, the system is used for multiplexing. During multiplexing, one or more other target types are detected from the same interrogation region at the same times. In some embodiments, this is accomplished by using different distinguishable intensities of the same two spectral bands. For example, Luminex beads that emit at up to ten different observable intensity levels can be purchased. By using up to ten different intensities in each of the two spectral bands already being imaged, up to 100 different target types can be distinguished. In other embodiments, more or fewer intensity levels are available and distinguishable. One circumstance mitigating the use of many different intensity levels, is the fact that observed intensity of specks of light from an optical marker varies as the optical marker moves in and out of the focal plane of the optics; so, an optical marker of fixed intensity may appear for some periods of time at lower intensity. This can be corrected somewhat by using maximum values of intensity for an individual that can be tracked from image to image.

In other multiplexing embodiments, additional excitation or detected spectral bands are introduced for one or more additional target types. For example, a second and third target type can be tracked by adding a third optical marker 240c excited or emitting/scattering in a third spectral band. A second target type can be tracked by binding with optical markers 240a and 240c with suitably chosen binding portions, while a third target type can be tracked by binding with optical markers 240b and 240c with appropriately chosen binding portions.

Thus, in some embodiments, providing the supply further comprises providing a supply of a multiplexed optical marker type that scatters or emits light under a corresponding different multiplexed condition and is configured to collocate with at least one other optical marker type at a single different target type. Serially imaging the interrogation region further comprises serially imaging the interrogation region under conditions that cause the multiplexed optical marker to emit light, to produce a multiplexed time series of images that detects light from the multiplexed optical marker type. A path of a speck of light from the multiplexed optical marker type is determined. It is determined whether the path corresponds to the multiplexed target type based on persistence of collocation of a speck of light from the at least one other optical marker type.

In steps 413 and 415, further inferences are made based on the number and timing of the paths of specks of light associated with the target types. For example, in step 413, the relative amount of one or more target types in the sample is determined based on the number of paths in a time period and the amount of fluid or other specks of light that passed through the interrogation region in that time. In some embodiments in which the amounts of the target types in the sample is known, step 413 includes producing a calibration curve that relates the number of paths per unit time to the concentration of the target types in the sample or the number of optical markers in the supply or some combination. In step 415, the characteristics of one or more target types are determined. For example if several samples are tested from subjects of different types, e.g., subjects with different conditions (e.g., symptoms or diseases) or the same subject at different times, then the concentration of the target types determined in step 413 can be used in step 415 to categorize the subject as having a particular condition or not or to characterize the effectiveness of a particular treatment.

Thus step 413 includes determining a calibration curve that relates the number of paths that correspond to the target type to concentration of the target type, when a concentration of the target type in the fluid is known.

Thus step 415 includes determining a characteristic of the fluid based on whether the path corresponds to the target type, wherein the characteristic is selected from a group comprising: presence of the target type, amount of the target type, relative amounts of multiple different target types, diagnosis of condition of subject which contributed a component of the fluid, and effectiveness of treatment given to the subject.

FIG. 4B is a flow diagram that illustrates a computation method 450 to perform step 411 of the method of FIG. 4A, according to an embodiment. Thus the method 450 is a particular embodiment of step 411. In step 451, multiple time series of images are obtained, each image representing light measured in an interrogation area of a fluid under conditions that cause only one optical marker type of at least two optical marker types to emit or scatter light. Each different time series of images indicates light measured from a different single optical marker type of the at least two optical marker types. The at least two optical marker types are configured to collocate with a single target type. Each image sequence contains data for thousands of unique specks of light from thousands of individual optical markers. Any method may be used to obtain the images, including retrieving directly from one or more detector arrays as an image detector, retrieving from local or network data storage, or scanning from hardcopies. Metadata about each image, such as the spectral channel represented and the time and experimental setup is also collected, again obtained in any fashion, including being entered manually by an operator.

In some embodiments, step 451 merely includes applying the alignment parameters determined earlier, e.g., in step 401. In some embodiments, during step 451, the alignment is refined of the views of the interrogation region at multiple imagers at the same time, each configured to receive in a different spectral band of optical wavelengths, so that different optical markers appear in different spectral bands. This step is performed to ensure that the images have not become misaligned. Typically, at least the channel edges can be aligned.

In step 453, a group velocity of the specks of light in the channel is determined, under one or more imaging conditions, e.g., in one or more spectral bands. For example, the current image in a first spectral band is cross correlated with the most recent preceding image, if any, at each of multiple shift and rotation positions, e.g., at one pixel along channel, two pixels along channel, three pixels along channel and one pixel cross channel, etc. The shift that gives the largest cross correlation is taken to be a measure of the group velocity. In some embodiments, this cross correlation is performed in subareas of the interrogation region so that a velocity field with potentially different values in different portions of the interrogation region is determined. In various embodiments, any known software package that provides this function can be used. For example, in some embodiments, after registration of the two image sequences for different spectral bands at one time, the algorithm proceeds in two main phases. The first phase quantifies local drift velocities using micron resolution particle image velocimetry (micro-PIV), known in the art.

In step 455, the location and intensity of an individual speck of light in a current image under first interrogation conditions are inspected to identify an optical marker associated with a current target type, for example based on size and intensity of specks falling within threshold levels. In an illustrated embodiment, the speck intensity and radius are evaluated using a non-linear Gaussian fitting routine, as illustrated, for example, in FIG. 1C. Thus, unique optical markers, e.g. emitting or scattering in one spectral band (called spectral channel 1 and abbreviated Ch 1), are identified, located and characterized. Any algorithm may be used. In an illustrated embodiment, this step is accomplished by the particle mask correlation and particle characterization (PMC-PC) method, known in the art and described in more detail below.

In step 457, a next path segment for speck of light from a first optical marker is determined based on the position of the speck in the previous image of the time series for the first interrogation conditions, such as for the first optical marker type 240a, based, at least in part, on the group velocity (e.g., velocity 312) determined in step 453. Any tracking method may be used. For example, in an illustrated embodiment described in more detail below, results of PMC-PC and micro-PIV are combined for a particle tracking velocimetry (PTV) subroutine enhanced by Kalman filter and χ⁻²-testing method (KC-PTV) and known in the art. This analysis results in accurate determination and tracking of the location of each speck of light over time and space.

In step 459, a collocation area is selected in a contemporaneous image under different interrogation conditions for a second optical marker, e.g., 240b, configured to be collocated with the target type. The collocation area is determined based at least in part on the size of the target type, as well as zero or more other factors, such as the optical size of the speck of light on the image detector, and the size of the optical marker types, and locations of the optical marker types on the target type. Thus, optical markers are determined to collocate with a single target type if the specks of light from the optical markers are located within a collocation distance that is based on size of the target type and size of the markers. In some embodiments, the collocation distance is based further on optical configuration and size of pixels in an imaging system used to perform the step of serially imaging the interrogation region.

In step 461, the maximum correlation is determined between the collocation area around the current position of the speck of light from the first optical marker the corresponding area in a contemporaneous image among a variety of shifts less than a threshold shift. In some embodiments, the threshold shift is selected so that a certain set of pixels around the speck of light from the first optical marker are involved in each correlation computation.

In step 471, it is determined whether the maximum correlation among the shifts exceeds a correlation threshold. If not, then control passes to step 481 to determine whether there is another interrogation condition (e.g., spectral band) to check. If so, then control passes back to step 459 to select the collocation area in a current image of the next time series collected under the next interrogation conditions for detecting specks of light emitted or scattered form the next optical marker.

If there is not another interrogation condition to check, then control passes to step 483 to determine whether there is another individual optical marker of the first optical marker type for which to determine the next path segment. If there is another individual optical marker of the first optical marker type, then control passes back to step 455 to determine the current position and intensity of the next optical marker of the first optical marker type based on the speck of light from the individual optical marker. Thus, in the time series of images, a plurality of paths are determined, each path for a speck of light from a different individual optical marker of a first optical marker type of the at least two optical marker types.

If there is not another individual optical marker, then control passes to step 485. In step 485, it is determined if there is another (multiplexed) target type to detect. If so, control passes back to step 453 or 455 to determine the group velocity or individual optical marker of at least one of the two or more optical markers that identify the next target type. If there is no other multiplexed target type, then control passes to step 487 to determine if there is another time with images in the time series of images. If not, the process ends. If so, then control passes back to step 451 to register the multiple contemporaneous images at the new time.

If it is determined, during step 471, that that the maximum correlation exceeds the threshold then, in step 473, the time since the last time series image is added to the persistence of the collocated specks. In some embodiments, the time averaged correlations is determined. In step 475, it is determined if the persistence exceeds the persistence expected of random motion superposed on the group velocity. If not, then control passes to step 481, described above, to check another interrogation condition to detect specks of light from another optical marker. If persistence exceeds the persistence of random motion, then in step 477 it is determined that the track represents the result of a deterministic binding event and therefore an instance of the target type, i.e., a target. Control then passes to step 481 described above to check another interrogation condition, if any, for specks of light from other optical markers. Thus, a number of the tracks that correspond to the target type is determined based on persistence of collocation of a speck of light from each of other optical marker types of the at least two optical marker types, which are different from the first optical marker type.

The described methods of FIG. 4A and FIG. 4B provide for target characterization based on persistent collocation of specks of light emitted from different optical markers in time series imagery. This method is applicable for either single time exposed images or sequential-in-time images of randomly distributed optical markers flowing in a channel. This method identifies and tracks hundreds of optical markers simultaneously and offers measurements of intensity, scatterer image size, and collocation between multiple colors (spectral bands) of specks of light emitted or scattered from the optical markers. The latter helps monitor labeled targets which emit at multiple wavelengths and scatterer-to-target type interactions. One unique feature of these techniques is that they can monitor the time evolution of particle-particle interactions. Tracking durations of about 8 seconds (s) have been demonstrated already; but, these durations can be increased by changing flow and/or imaging conditions.

Further, as described in more detail below for some embodiments, the described image analysis can handle suspensions with speck densities in excess of 10⁸ specks per milliliter (mL, 1 mL=10⁻³ liters), expressed in units of mL⁻¹. By comparison, FCM require densities of 10⁶ mL⁻¹ or less. All of the analysis can be performed on a target and optical marker suspensions inside virtually any microchannel or tube with optical access for imaging, including off-the-shelf microfluidic chips. In comparison, FCM and LSC systems each require specialized particle handling systems: Precise hydrodynamic focusing for FCM and immobilization and scanning for LSC.

### 2. Example Embodiments

### Experimental setup

As used herein one or more target types are identified by the location of specks of light emitted or scattered from two or more optical markers. The optical markers emit or scatter light either spontaneously or in response to excitation of some kind. What is detected by the imaging system is specks of light from the optical markers when excited, if applicable.

According to an example embodiment, the method is optimized for analyzing specific scatterer-to-particle interactions and the resulting collocation and correlation of speck motions. The example setup used a standard epifluorescence microscope with a 20× objective lens (e.g., as the first lens 110 at the top of FIG. 1A); a CCD camera equipped with a dual-view color separator (as one beam separator 112 and two filters 114 and image detector 116); and a mercury arc lamp for illumination (as excitation source 122). The particles (including targets or optical markers or some combination) are suspended in an aqueous solution and loaded onto a microfluidics chip (as channel 120). The particles are propelled through the interrogation region via electrophoresis. The example instrumentation is simple and off-the-shelf, and the method relies on sample preparation and image analyses and computations for detailed analysis. The custom particle tracking algorithm tracks each speck of light from an optical marker type and monitors its location, size, diffusivity, optical intensity, and its correlation to speck images at other interrogation condition (such as detection wavelengths) in time. The technique can generate time resolved measurements for thousands of particles in minutes.

A solution containing fluorescent red and green beads and probes was mixed with target DNA in a hybridization buffer. A bead doublet is formed when Probe 1 on red fluorescence bead (Bead 1) label, and Probe 2 on green fluorescence bead (Bead 2) label are hybridized to the target DNA sequence. The bead-DNA mixture was electrophoresed in a channel with a transparent top wall and visualized using a microscope equipped with a dual-view system and high-sensitivity CCD camera.

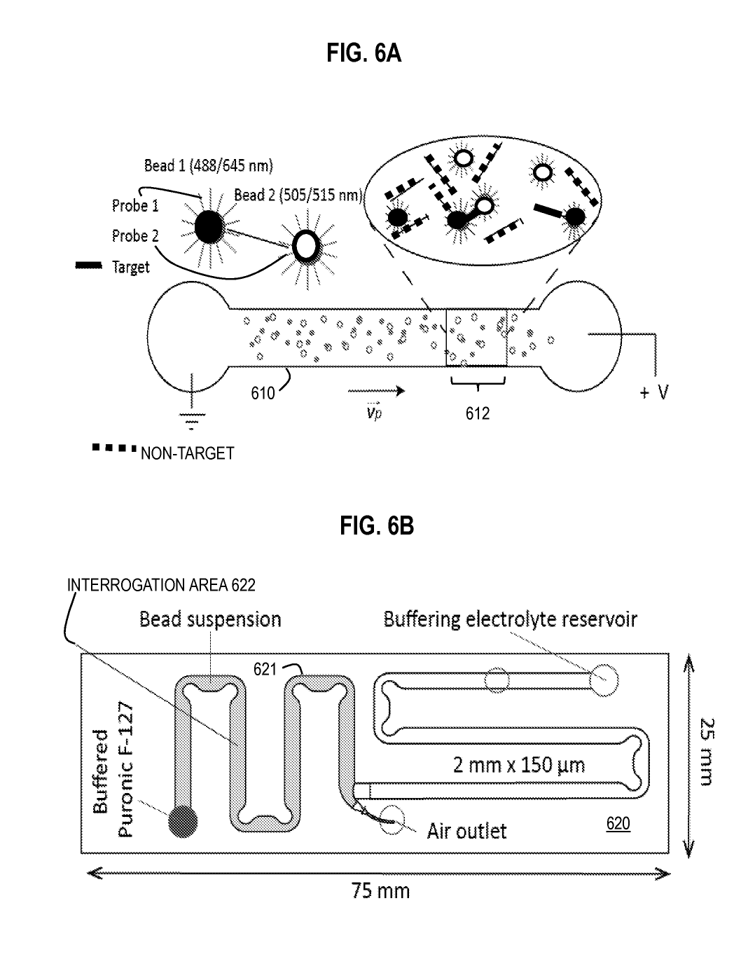

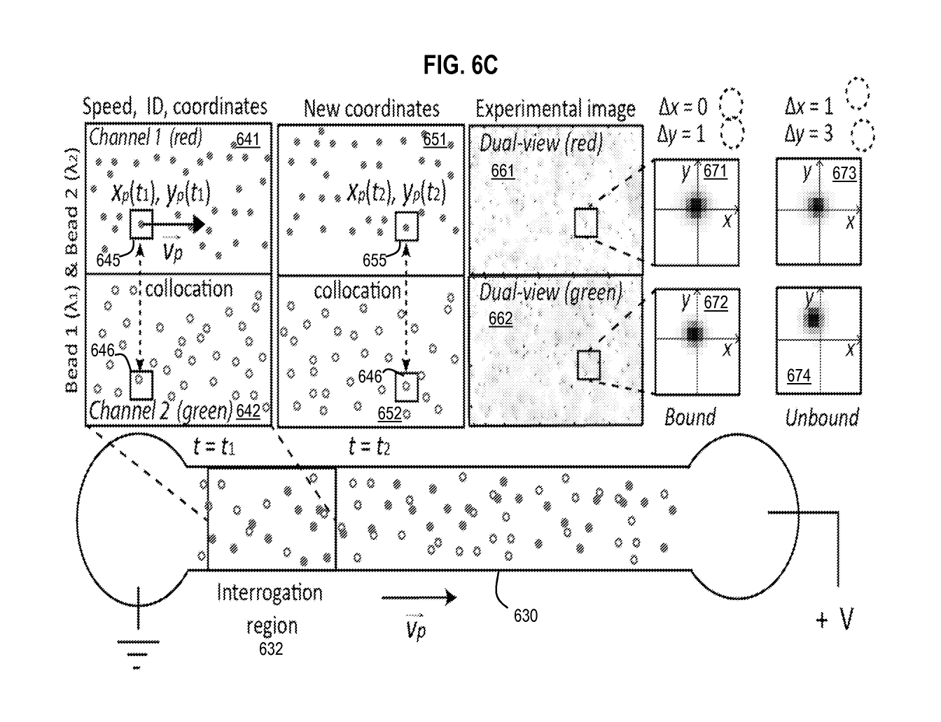

FIG. 6A through FIG. 6C are a block diagram that illustrate an example experiment with beads as optical markers and a DNA type as a target type, according to an embodiment. FIG. 6A is a block diagram that illustrates a fluidic channel 610 in which fluid is propelled by electrophoresis imposed by a voltage difference V between an input port (e.g., at electrical ground) and an outflow port (e.g., at a voltage of +V). This voltage difference causes particles to move with a velocity νp. The interrogation area 612 is disposed before the fluid encounters the exit port.

In FIG. 6A, the target DNA molecule type is represented by a thick solid line and non-targets (including other DNA molecule types) are represented by a thick dashed line. Two beads are used. Fluorescent bead 1 (closed circle) is excited by light at 488 nm and emits light at 645 nm and includes probe 1 that binds with a first portion of a target DNA molecule type. Fluorescent bead 2 (open circle) is excited by light at 505 nm and emits light at 515 nm and includes probe 2 that binds with a different second portion of the same target DNA molecule type. The closed circles in channel 610 represents example locations where specks of light from beads of type bead 1 are observed; and, the open circles in channel 610 represents example locations where specks of light from beads of bead 2 are observed. The inset oval suggests that some of the beads 1 and 2 with corresponding probes 1 and 2 are not bound to a target DNA molecule and one of each is bound to one target DNA molecule. One target DNA molecule is illustrated bound to bead 1 but not to bead 2.

In experimental embodiments, beads of type bead 1 comprise FluoSpheres® microspheres from Molecular Probes Inc Life Technologies of Grand Island, N.Y., with Label (Excitation wavelength/Emission wavelength): Yellow-green (505/515), Catalog number: F8823, Nominal bead diameter: 1.0 μm, Coupling surface: Carboxylate, solids: 2%, DNA probe (desalted): 5′-Amino Modifier C12-CACAAAGTGGTAAGCGCCCTC (SEQ. ID NO. 1). Beads of type bead 2 comprise FluoSpheres® microspheres from Molecular Probes Inc Life Technologies of Grand Island, N.Y., with Label (Excitation wavelength/Emission wavelength): Crimson-fluorescent (625/645), Catalog number: F8816 Nominal bead diameter: 1.0 μm Coupling surface: Carboxylate Solids: 2% DNA probe (desalted): 5′-Amino Modifier C12-CGGATTGGAGTCTGCAACTCG (SEQ. ID NO. 2). Beads were functionalized with Amine modified DNA probes by Radix Biosolutions of Georgetown, Tex., and suspended in Tris-EDTA, pH 8 at 2% solids. The desalted DNA probes and PAGE purified DNA targets were purchased from Integrated DNA Technologies (IDT) of Coralville, Iowa. The target DNA type had a sequence AAACGAGTTGCAGACTCCAATCCGAAAAGAAGTAGGTAGCTTAACCTTCGGGAG GGCGCTTACCACTTTGTGTTT (SEQ. ID NO. 3).

Because the beads used in this assay were carboxylate modified (negatively charged at the operating pH of 8), they electrophorese in the presence of an applied electric field. The beads were suspended freely in solution, so that Brownian motion separates randomly collocated beads, and so distinguish such randomly collocated beads from beads which are deterministically collocated because bound via a DNA target type to form a doublet of optical makers. Note that the extinction coefficient for polystyrene beads in the wavelength range of 300 nm to 1200 nm is less than 10⁻⁵. Beads are therefore approximately transparent to the excitation and emission wavelengths, so that overlapping of two particles along the optical axis is not expected to result in significant signal attenuation due to absorption.

FIG. 6B is a block diagram that illustrates an example poly-methyl methacrylate (PMMA) microfluidic chip, 620, suitable for performing an embodiment. The chip 620 is 25 millimeters (mm, 1 mm=10⁻³ meters) wide by 75 mm long and includes a microchannel 621 with an interrogation area 622 imaged by a charge coupled device (CCD). The microchannel 621 with dimensions 100 mm long by 2 mm wide by 0.15 mm deep is loaded with the buffered bead suspension. The buffered bead suspension contained 20 milliMolar (mM, 1 mM=10⁻³ Molar) Tris, 10 mM hydrochloric acid, HCl, 0.08% Triton X-100, 50 mM sodium chloride, NaCl, 10 nanoMolar (nM, 1 nM=10-9 Molar) target DNA, and 3×10⁸ beads/milliliter (mL, 1 mL=10⁻³ liters) of each color. The solution was pressure loaded into the microfluidic chip. End-channel reservoirs and one mid-channel reservoir were filled with approximately the same volume of aqueous buffer solution (1 M Tris, 500 mM hydrochloric acid, HCl) in attempt equalize hydrostatic pressure and minimize pressure driven flow. To reduce unwanted pressure-driven flows in the channels, pluronic-F127 was used as a phase change material to seal off the buffering well. The mixture of pluronic-F127 is a liquid at low temperature and solidifies into a gel at room temperature.

Platinum electrodes were placed in the loading and output well and electrophoresis was initiated by applying a current of 100 microAmperes (μA, 1 μA=10⁻⁶ amperes) across the electrodes.

The bead suspension is imaged using an Olympus IX70 microscope from Olympus Corporation of America of Center Valley Pennsylvania, equipped with a 20×, NA=0.5 Olympus UPlanFl objective, a 16-bit CCD (Cascade 512F) camera (520×520 of 16 μm pixels). The optical depth of field of the microscope objective is 3.8 μm, evaluated at wavelength (λ)=550 nm. The characteristic depth of the particle tracking measurement volume is 11.5 μm (at λ=550 nm). The focal plane was placed about halfway between the bottom and top of the channel 621 in the microchip 620. The interrogation region comprises a 150 μm by 275 μm field of view, roughly centered along a spanwise 2 mm width of the channel. Spatial separation of two wavelengths was achieved using an XF53 dual pass filter cube (Omega Optical) with peak excitation wavelength ranges of 475 nm to 500 nm and 550 nm to 600 nm, and peak emission wavelength ranges of 500 nm to 550 nm and 600 nm to 675 nm, in combination with a quad-view imager (Micro-Imager, Photometrics, Tucson, Ariz.). In a typical experiment, 200 images of particles are recorded illuminated with a mercury light source for 5 milliseconds (ms, 1 ms=10⁻³ seconds) at a frequency of 1 Hz. During this time, on the order of 1,000-10,000 unique beads traverse through the field of view. The particle motion and imaging rate can be increased for applications requiring higher throughput and where particles or cells require shorter monitoring times.

FIG. 6C is a block diagram that illustrates example imagery captured during an experimental embodiment, such as from microchip 620, according to an embodiment. Scatterer imaging, tracking and collocation for scatterer monitoring and scatterer-to-particle binding assays are depicted. A two-color version is shown here, but the system is easily scalable to four colors using off-the-shelf instrumentation. A typical experiment involves (1) loading a solution containing particles emitting in the red and green into a microchannel; (2) electrophoresing the particles through a detection region with optical access; and (3) imaging at a user-specified rate using a microscope equipped with dual-view system and high-sensitivity CCD camera. The dual-view system chromatically separates the particle images into separate spectral channels on separate spatial domains on the CCD array. The method 450 determines location and in-plane velocity vectors of each scatterer in one spectral channel. This analysis is used to track the coordinates, image size, and fluorescence intensity of the individual scatterers in time. The subregions serving as collocation areas surrounding the scatterers in the first spectral channel (Ch 1) are identified and tracked then cross-correlated with the corresponding subregions in the other spectral channel, (Ch 2). The persistence (in time) of a high cross-correlation signal indicates deterministically collocated scatterers bound to targets.

As shown in FIG. 6C, fluidic channel 630 includes an interrogation area 632, where scatterers above a certain size and intensity are detected in two images 641 and 642 taken in two corresponding spectral channels Ch 1 (red) and Ch 2 (green) at a first time, t1. The location coordinates Xp(t1), Yp(t1) of an individual scatterer is determined in Ch 1 (as well as intensity and size of the scatterer) and a collocation area 645 is centered on the coordinates of the scatterer. The corresponding collocation area 646 is determined in the other channel, Ch 2. If an scatterer above a certain size and intensity is also seen there, as indicated by a correlation above a certain threshold, then a candidate doublet has been found. The locations of other scatterers in Ch 1 are determined and a collocation area centered on each. Each collocation area is examined in the image of Ch 2 to find other candidate doublets for other instances of the same DNA type. If any doublets persist longer than Brownian motion, then that persistent doublet indicates an instance (molecule) the target DNA type.

Scatterers are detected in two images 651 and 652 taken in the spectral channels Ch 1 (red) and Ch 2 (green) at a later time, t2. The location coordinates Xp(t2), Yp(t2) of the same individual scatterer is determined in Ch 1 based on the group velocity of the particles νp; and, a collocation area 655 is centered on the new coordinates of the same scatterer. The corresponding collocation area 656 is determined in the other channel, Ch 2. If a scatterer is also seen there, by having a correlation greater than the threshold, then the doublet has persisted for the time t2−t1. If that time is greater than the persistence expected from Brownian motion, as indicated by exceeding a temporal threshold, then a molecule of the target DNA type has been found.

Experimental images 661 and 662 of the interrogation area in two spectral channels, Ch 1 and Ch 2, respectively, are also shown in FIG. 6C. The x and y coordinates are centered on the position of the first scatterer. The second scatterer in Ch 2 within the collocation area is shown as graph 672. If the specks were located near the center of the coordinate system, then the doublet persists. For example, the intensity peak in 672 is centered on the same x (Δx=0) and is off the same y by only one pixel (Δy=1). This is considered a doublet suggestive of binding, and a candidate for an instance of the target if it persists. In contrast, a different scatterer shows an intensity mapped in graph 673, and a peak intensity at the other channel mapped in graph 674. The intensity peak in graph 674 is highest at a Δx of 1 and Δy of 3, too far to be considered a candidate doublet; and, thus the two scatterers are determined to be unbound (not bound to a target).

The two spectral channels are first spatially registered using registration parameters (e.g., shift and rotation) based on a bright field image of an alignment mask (e.g., as shown in FIG. 5), as determined again or earlier. The numerical processing steps described above in FIG. 4B were then performed in a post processing mode, after collection of all the time series images. Each image sequence contains data for thousands of unique scatterers. After registration of the two images, the algorithm proceeds in two main phases. The first phase quantifies local drift scatterer velocities using micron resolution particle image velocimetry (micro-PIV). Unique scatterers in Channel 1 (Ch1) are then identified, located and characterized via the particle mask correlation and particle characterization (PMC-PC) method. The scatterer intensity and radius are evaluated using a non-linear Gaussian fitting routine. The algorithm then combines results of PMC-PC and micro-PIV for a particle tracking velocimetry (PTV) subroutine enhanced by Kalman filter and χ²-testing method (KC-PTV). This analysis results in accurate determination and tracking of the location of each scatterer over time and space. The second phase of the algorithm cross-correlates collocations areas (also sometimes called subregions) surrounding the particles' locations identified in Ch1 with corresponding subregions of the same or different size in the registered Ch2. Ch2 scatterer characteristics, such as radius and total fluorescence, are evaluated using the Gaussian fitting subroutine. Thresholds for intensity, size, velocity, and correlation coefficient are applied at each step to eliminate spurious results.

### Scatterer Imaging

Various embodiments utilize speck brightness patterns, and so imaging conditions are recommended such that speck image diameters correspond to distances of 3 or more pixels (roughly 3 to 8 pixels are preferred). To discriminate between bound and closely neighboring but unbound optical markers, the persistence (in time) of the spatial correlation of specks is monitored. This option places a preference for a minimum amount of time particles are tracked. The particles are advantageously tracked long enough for Brownian motion or other dispersion to cause separation of optical markers unbound to targets. For particle separation phenomena determined by Brownian motion, this minimum time of observation can be estimated from particle diffusivity. For particles with radius, rₚ, and speck radius (geometrically projected into the object plane), rₚᵢ, Equation 1 is advantageous to estimate a minimum time, t_{ch}, over which to track optical marker pairs undergoing Brownian diffusion:

$${L}_{ch} = \sqrt{4 {D}_{eff} {t}_{ch}} > [ ( {r}_{p , 1} + {r}_{p , 2} ) + ( \frac{5}{4} ) min \{ {r}_{pi , 1} , {r}_{pi , 2} \} ] \qquad ( 1 )$$

Here, L_{ch} is a characteristic minimum optical markers center-to-center distance in object space required before the algorithm concludes the particles are not bound. At the characteristic time, t_{ch}, diffusion statistics suggest that 67% of all randomly aligned particles pairs with effective diffusivity of D_{eff}, are separated by L_{ch}. The Brownian separation distance between two particles is estimated using an effective diffusion coefficient of the form D_{eff}=Dₚ₁+Dₚ₂, where Dₚ₁ and Dₚ₂ are, respectively, the diffusivities of particles imaged in spectral channels 1 and 2. Here it is assumed that the diffusive motions of the closely-spaced particles are statistically independent. Combining equation (1) with the Einstein diffusivity expression, the characteristic minimum evolution time can be solved using Equation 2.

$${t}_{ch} = \frac{3 π {μ [ ( {r}_{p , 1} + {r}_{p , 2} ) + ( \frac{5}{4} ) min \{ {r}_{pi , 1} , {r}_{pi , 2} \} ]}^{2}}{2 kT \{ \frac{1}{{r}_{p , 1}} + \frac{1}{{r}_{p , 2}} \}} \qquad ( 2 )$$

In the experimental embodiments, this time is roughly 2.5 seconds. Appropriate choices for monitoring time are discussed further below.

The persistence (in time) of a high cross-correlation signal indicates deterministically collocated specks of light from optical markers and hence optical markers bound to targets. This is in contrast to unbound, but closely neighboring specks of light from optical markers, which eventually separate due to Brownian motion and/or dispersion. Paired specks from the two spectral channels in the example embodiment are identified as bound only if their maximum correlation at each time step t, called collocation coefficient and signified as R_{12,max}(t), remains sufficiently high for longer than a predefined minimum tracking time based on t_{ch} determined from Eq. 2. Speck pairs in Ch 1 and Ch 2 with collocation coefficient traces, R_{12,max}(t), with a median collocation value above a predefined threshold, \tilde{R}₁₂, are determined to be bound. Appropriate choices for median collocation thresholds, \tilde{R}₁₂, are discussed in more detail below.

To prevent highly correlated, non-particle related events from contributing to bound event counts, an intensity-based threshold filter has also been implemented, which removes all collocated specks of light with intensities 3 (for simulated) and 1 (for experimental data) standard deviation away from the mean of the population of “bound” specks.

### Micron-Resolution Particle Image Velocimetry (Micro-PIV)

Measurements of spatially correlated speck motion in one interrogation condition, such as one spectral channel (e.g., velocities averaged over finite sub-areas of the interrogation area containing multiple specks) were used to guide tracking of individual specks under one interrogation condition (e.g., in one spectral channel). This correlated speck motion is the result of non-Brownian transport such as fluid flow or electrophoresis or both. Micro-PIV provides a robust and high-resolution method for determining such spatially correlated speck velocities. Micro-PIV was developed specifically for microfluidic applications, and has been reviewed and described extensively. The process limits speck tracking to specks near the focal plane of epifluorescence imaging. The standard process measures the x- and y-components of the velocity field in the imaging plane. For the micro-PIV implementation in some embodiments, 30 by 100 pixel sub-areas were used with 50% overlap (for a total of 25 sub-areas). Since the flows here were approximately steady, velocity information were typically averaged by ensemble averaging 50 correlation functions (each associated with an image pair spanning two different times in one spectral band) per velocity calculation. For the experimental data described herein, over 200 correlation functions were ensemble averaged.

### Particle Mask Correlation (PMC)

PMC is performed in parallel with micro-PIV analysis. The PMC method identifies particle images (specks) and their coordinates by convolving raw images with a kernel “mask” made up of a two dimensional circularly symmetric Gaussian brightness pattern, Iₘ, as illustrated in FIG. 1C and expressed by Equation 3.

$${I}_{m} ( x , y ) = A exp [ - \frac{{( x - {x}_{0} )}^{2} + {( y - {y}_{0} )}^{2}}{2 {σ}_{m}^{2}} ] \qquad ( 3 )$$

The peak brightness, A, is set to unity, but the value is arbitrary as cross-correlations between the image and particle mask are normalized, as given in Equation 4, below. The mask standard deviation σₘ is chosen equal to or smaller than the radius of the smallest expected speck radius in the image set. In the experimental embodiments, the speck brightness spans approximately 3 pixels, and so σₘ=lₚ, where lₚ is the pixel dimension in the object plane.

The particle mask is scanned over the entire image plane and the normalized cross-covariance coefficient, r_{PMC}, is calculated at each pixel location, (xₒ, yₒ). The normalized cross-covariance coefficient between the particle mask centered at (xₒ, yₒ) and the image subregion of same size centered at (xₒ, yₒ) are computed as given by Equation 4.

$${r}_{PMC} ( {x}_{0} , {y}_{0} ) = \frac{{\sum }_{i = {x}_{0} - m / 2}^{{x}_{0} + m / 2} {\sum }_{j = {y}_{0} - n / 2}^{{y}_{0} + n / 2} ( I ( i , j ) - \hat{I} ) ( {I}_{ma} ( i , j ) - {\hat{I}}_{ma} )}{\begin{matrix}\sqrt{{\sum }_{i = {x}_{0} - m / 2}^{{x}_{0} + m / 2} {\sum }_{j = {y}_{0} - n / 2}^{{y}_{0} + n / 2} {( I ( i , j ) - \hat{I} )}^{2}} \\ \sqrt{{\sum }_{i = {x}_{0} - m / 2}^{{x}_{0} + m / 2} {\sum }_{j = {y}_{0} - n / 2}^{{y}_{0} + n / 2} {( {I}_{ma} ( i , j ) - {\hat{I}}_{ma} )}^{2}}\end{matrix}} \qquad ( 4 )$$

Here I(i,j) is the brightness value of the image plane at (i,j). In this study, the particle mask size (n,m) is set to 8σₘ×8σₘ. The value of r_{PMC} varies between −1 to 1, depending on the degree of similarity between the brightness patterns. Pixels with high covariance coefficients indicate the presence of specks. Using a covariance threshold of 0.7 enables the PMC method to find all concentric convex brightness patterns of size of roughly <8 σₘ. The calculated covariance coefficient plane is transformed to a binary image using this threshold. For simplicity, the pixel with the highest r_{PMC} value is assigned as the coordinate for the center of the particle, (x_{p,1}, y_{p,1}).

As an approximation, the speck radius (geometrically projected into the object plane) is evaluated as r_{pi,1}=\sqrt{(glₚ)²/π}, where g is the number of ‘unity’ pixels for a particle group in the binary image, and lₚ is the size of the pixel in the object space. While the cross-covariance was performed on raw images, the total, or integrated speck intensity, I_{p,1}, is estimated by summing the background corrected intensity of a 4r_{pi,1}×4r_{pi,1} subregion centered at the scatterer center (x_{p,1}, y_{p,1}). The corrected images are evaluated as given by Equation 5.

$${I}_{C} = \frac{{I}_{raw} - {I}_{bg}}{{I}_{flat}} \qquad ( 5 )$$

The background of the raw image, I_{bg} is obtained by filtering the original image with a median filter of size 10σₘ×10σₘ. The flatfield, I_{flat}, is obtained by imaging the microchannel interrogation region filled with uniform concentration of dyes (1 μM AF488 and 1 μM AF647) which are processed with a median filter of size 10σₘ×10σₘ.

This method can significantly improve the accuracy of scatterer localization and size and fluorescence characterization by using a sub-pixel resolution method. The sub-pixel resolution approach fits the scatterer brightness pattern in the local 8σₘ×8σₘ subregion of background corrected particle image using a non-linear Gaussian fitting routine.

### Particle Characterization (PC)

PC is performed to significantly improve the accuracy of particle localization and the estimates for speck radius, rₚᵢ and integrated speck image intensity, Iₚ. In this routine a two dimensional circularly symmetric Gaussian brightness pattern, I_{P}, is used to fit the speck brightness patterns in the corrected images using Equation 6.

$${I}_{P} ( x , y ) = A exp [ - \frac{{( x - {x}_{p} )}^{2} + {( y - {y}_{p} )}^{2}}{2 {r}_{p}^{2}} ] + b \qquad ( 6 )$$

The Gaussian fitting is performed on an 8σₘ×8σₘ subregion bounding the speck. As a first guess, the routine uses the median intensity of the subregion for the background, b; the highest pixel intensity minus the background for the amplitude, A; and the simple estimates, described above, for particle coordinates and speck radius for xₚ, yₚ and rₚᵢ. This fitting routine was used for more detailed cytometry-like data of particle populations. For the simulated particle-to-particle binding assay data presented below, this Gaussian fitting algorithm step was disabled in order to save computational time. To eliminate large aggregates and out-of-focus optical markers from analysis, size- and intensity-based threshold filters were implemented. Specks with too small an intensity or too large a size were excluded as candidates for being collocated with a target type. For the particle-to-particle collocation analysis presented in this study (unless stated otherwise), the size-based threshold was set to eliminate specks with radii larger than the mean plus 2 times the standard deviation of the speck population in each image. The intensity-based threshold eliminated all features with intensity 3 times the standard deviation away from the mean of the speck population, both too bright and too dim. The output parameters of these phase includes particle coordinates, x_{p,1}, y_{p,1}, speck radius, r_{pi,1}, and integrated background-corrected total scatterer fluorescence intensity, Iₚ, for each speck in the image sequence.

### Kalman Filter and Chi Squared Enhanced Particle Tracking Velocimetry (KC-PTV)

In this phase, speck identities between consecutive images are correlated and matched in order to track their motion in time. For robust and accurate operation, a Kalman filter and χ²-test is used to track each speck representing a unique optical marker over time and space in one spectral band. This method, KC-PTV, was developed by Etoh and Takehera for particle tracking velocimetry (see Takehara, K. and Etoh, G. T. Journal of Visualization, 1999, vol 1 (no. 3), pp 313-323). KC-PTV was later adapted for microfluidics by incorporating micro-PIV. In KC-PTV, speck data (here location and speck radius) from one time step (t=0) is used to predict the speck information in a next time step (t=t+Δt). The probability of two specks belonging to the same optical marker identity is evaluated using a χ²-test which uses the image data as parameters.

In an experimental embodiment, filters were applied to the data which reject speck motions which are significantly far from bounds determined by local micro-PIV velocity data and particle diffusion estimates. For example, speck matches are rejected with apparent velocities, ν_{p,a}, which fail the following criteria:

5v_{diff}>|v_{p,a}−v_{PIV}(x_{p,1},y_{p,1})|.

Here, v_{diff}=\sqrt{4Dₚ/Δt}, and v_{PIV}(i,j) is the drift velocity at the particle position (x_{p,1},y_{p,1}) obtained from micro-PIV analysis. Output parameters from this phase, including optical marker identification number (ID), and the corresponding speck track coordinates, x_{p,1}(t), y_{p,1}(t), speck radius, r_{pi,1}(t), and integrated background-corrected speck fluorescence intensity, Iₚ(t), are stored for further processing.

### Collocation in Different Spectral Channels

An example optical marker collocation approach begins by positioning a small 8σₘ×8σₘ subregion (the 8σ subregion) at the coordinates of each optical marker speck identified and tracked in Ch 1. The algorithm then selects a 16σₘ×16σₘ subregion (the 16σ subregion) in Ch 2 centered at the same image coordinates. The Ch 2 16σ subregion is an example of the collocation area of FIG. 4B. The shifts, for which correlations are computed, are limited so that the Ch 1 8σ subregion always completely overlaps the Ch 2 16σ subregion; thus eliminating the known biases associated with cross-correlations of finite-sized correlation functions.

For the collocation analysis, the normalized cross-covariance is evaluated as described by Eq. 4, but in which Iₘ is set to the Ch 1 8σ subregion and I is set to the Ch 2 16σ subregion. The parameter r_{PMC} is then interpreted as the degree of correlation in the position of the specks detected in both Ch 1 and Ch 2, which is referenced as collocation coefficient, R₁₂(x₀,y₀,t). R₁₂(x₀,y₀,t) is evaluated for window offsets (also called x and y shifts) of (xₒ, yₒ) equal to or less than a predefined value, Sh=(r_{p,1}+r_{p,2})+(1/4) min{r_{pi,i},r_{pi,2}}. The offset (xₒ, yₒ) with the highest R₁₂(x₀,y₀, t) value is initially assigned as the coordinate for the center of the Ch 2 particle, (x_{p,2}, y_{p,2}) and the collocation coefficient there, R_{12,max}.

If the maximum coefficient, R_{12,max}, is below 0.6 (e.g., as determined in step 471 of FIG. 4B), then it is concluded that no speck is collocated in the Ch 2 16σ subregion. For such a case, r_{pi,2} is set to zero and I_{p,2} is estimated in Equation 5 as the sum of the background corrected scatterer image intensity of a 4r_{pi,1}×4r_{pi,1} subregion centered at (x_{p,1}, y_{p,1}) in Ch 2.

If R_{12,max} is above 0.6, it is concluded that there is a collocated speck in the Ch 2 16 subregion. To estimate the radius of the speck, the collocation matrix is converted to a binary image and the group of pixels having value 1 is found and associated with the Ch 2 particle coordinates (x_{p,1}, y_{p,1}). The speck radius is estimated as r_{p1,2}=\sqrt{(glₚ)²/π}. Speck radius is subsequently used to determine the Ch 2 speck intensity, I_{p,2}, by summing the background corrected image intensity of a 4r_{pi,2}×4r_{pi,2} subregion centered at (x_{p,2}, y_{p,2}) using Equation 5.

### Monte Carlos Simulations

Monte Carlo simulations, a well-accepted method of evaluating particle tracking algorithms, were used to guide choices of user specified parameters and to demonstrate the performance and robustness of the target characterization based on speck collocation persistence. Monte Carlo simulations were performed of Brownian particles with Gaussian brightness patterns for specks of one pixel standard deviation. The particles were randomly distributed in a 500×150 interrogation area simulation domain which corresponded to a CCD pixel array. Particle diffusivity was set to 0.44 μm²/s, and the particles were given a uniform advective velocity of ν_{p,x}=10 μm/s. As typical with Brownian Monte Carlo, specular reflection boundary conditions were chosen for the side walls of the simulation domain. Further, particles exiting the x=150 end of the domain were reintroduced at x=0, at random y-coordinates. To explore the sensitivity of the method 450 to experimental conditions, the following variations were simulated: (1) percentage of collocated specks (both optical markers bound to a target) between the two spectral channels ranged between 0-100%, (2) distance between specks varied between 5 to 20 μm, and (3) image signal to noise ratio (SNR) ranged between 2 to 100. Note that the average inter-speck distance of nearest neighbor specks in 2D can be determined as L_{IP}=0.5 η^{-1/2}, where η (in units of m⁻²) is speck area density. Inter-speck distance of 5 to 20 μm is equivalent to about 800 to 50 specks in a 500×150 simulation domain.

The probability of successful optical marker identification and collocation relies on the degree of correlation between two normalized brightness patterns. Signal-to-noise ratio (SNR) has a strong effect on the brightness pattern of raw images. SNR is defined here as the peak speck intensity above the mean of local background divided by 2 times the standard deviation of background image intensity. To determine how SNR influences speck tracking and collocation accuracy, Gaussian white optical noise was added to the simulated speck images resulting from the particle fields. During the 200 second (s) simulation time, 1422 unique particles were introduced, 1200 of which had a residence time of roughly 15 s.

Speck tracking was performed on the simulated images and the histogram of tracked speck times as a function of SNR was plotted. In the case of low image SNR (SNR=2), over 200 s, approximately 5000 specks from individual optical markers were identified, but only 10% were tracked for 3 s or longer. Note, that when the algorithm loses a speck from a particular optical marker due to the interfering effects of SNR, then, if a speck from the same optical marker reemerges from the noise at a later time step, the speck is identified as coming from a new optical marker (as would happen during an experiment). Consequently, the counts of the number of optical markers increases, and tracked speck times shorten with increasing image noise. Consistent with this is the large discrepancy present between the number of optical markers detected (5000) and simulated (1400) at SNR=2. For image SNR of 5 or above, the number of optical markers identified and their tracked times converges to the known values.

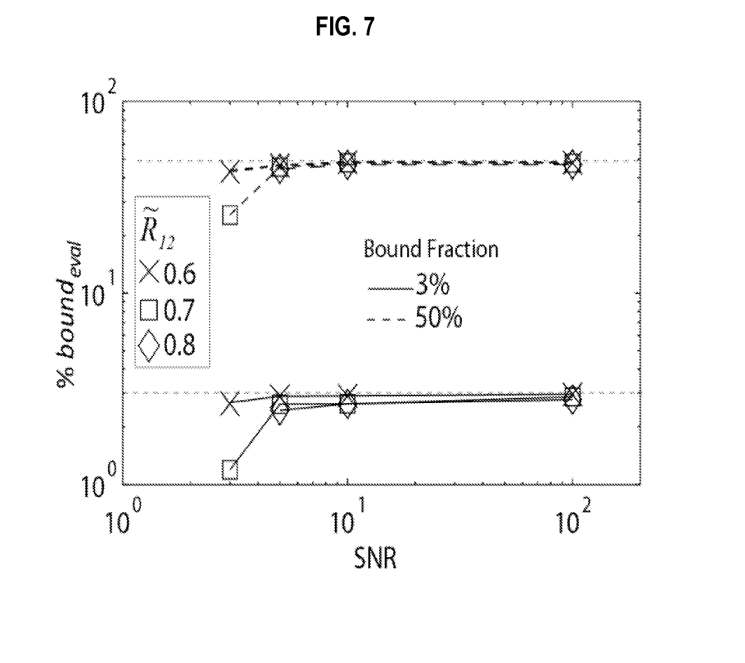

FIG. 7 is a graph 700 that illustrates example dependence of performance on signal to noise ratio (SNR), according to an embodiment. The horizontal axis 702 indicates SNR (dimensionless); and, the vertical axis 704 indicates the fraction of specks collocated in the two spectral bands (considered bound optical markers) in percent. One set of traces 706 is for simulations in which 3% of the optical markers are collocated in both spectral bands; and, the other set of traces 708 is for simulations in which 50% of the optical markers are collocated in the two spectral bands (considered bound). In FIG. 7 is shown the combined accuracy of speck tracking and collocation as a function SNR and collocation coefficient threshold, \tilde{R}₁₂, for two different simulation fractions of bound particles. Three different values of \tilde{R}₁₂, are plotted in each set of traces, 0.6 indicated by crosses, 0.7 indicated by open squares, and 0.8 indicated by open diamonds.

For both sets of traces 706 (3% bound optical markers) and 708 (50% bound optical markers), increasing image SNR improves collocation accuracy. In general, strong image noise results in an underestimation of the bound fraction. For SNR=2, no bound optical markers were detected for any collocation thresholds in the tested range (\tilde{R}₁₂=0.6 to 0.8).

The collocation results for images with SNR=3, using threshold of \tilde{R}₁₂=0.6 are in close agreement with the simulated values, but raising \tilde{R}₁₂ to 0.8, causes complete failure of the collocation analysis.

For SNR >5, the collocation accuracy is weakly dependent on SNR and collocation thresholds. For example, prediction of the 3% bound fraction case (solid line) at SNR=10, is 2.91%, when \tilde{R}₁₂=0.6, and 2.64%, when \tilde{R}₁₂=0.7 to 0.8. Based on these analyses, it is determined that a value of \tilde{R}₁₂ about 0.6 yields an appropriate accuracy for a fairly wide range of image SNR.

Results of collocation analyses performed with 0% simulated bound particle fraction were used to study the limit of detection (LOD) in absence of collocated optical markers bound to targets and to quantify false positive rates in negative controls. For example, at image SNR=100, the example embodiment found that speck doublets, indicative of optical markers bound to targets, had false detection rates of 0.9%, 0.6%, and 0.25% of the total specks for collocation threshold values of 0.6, 0.7 and 0.8, respectively. False positive collocation detections occur in the negative control case due to failure of the speck intensity threshold filter originally implemented in the collocation phase of some simulation embodiments. This intensity based threshold relies on the intensity distribution of true positive speck matches in Ch 2. Since there are no true positive matches in the negative control case, the intensity distribution obtained this way is not a true representation of the Ch 2 speck intensities. These bound fractions, therefore, represent the limit of detection (LOD) of embodiments in the absence of a priori Ch 2 scatterer intensity calibration. To improve the LOD, the evaluation of scatterer intensities in Ch 2 using the PMC-PC method is advantageous. The calibrated intensity distribution can then be used to filter speck matches in the collocation phase. After a simple calibration of Ch 2 specks was performed, the example embodiment detected 0% bound fractions for all collocation thresholds in the negative control case, thus eliminating false positives.

The rate of analysis of an example collocation method is primarily limited by two parameters, the maximum speck concentration tolerated by the method and the minimum monitoring time, tₘ. Here the effect of these parameters on speck tracking and collocation performance is examined. Image pairs containing 50, 100, 200, 400 and 800 specks per image, 3% of which were deterministically collocated (e.g., simulating both optical markers bound to a target). During the 200 s simulation time, 718, 1437, 2851, 5692 and 11314 unique optical markers were introduced, most of which had a residence time (time spent in interrogation region) of roughly 15 s. A histogram of tracked speck times (duration over which algorithm tracks each speck) as a function of density (per unit area) of specks. At high speck area density, neighboring specks can influence each other's brightness patterns, so the success rate of tracking, and thus the tracked speck times, decrease. For image sets containing 200 or more particles, the tracked speck times are limited by speck crowding, and not by the user-specified parameters of velocity and interrogation region size. For example, for the highest speck density case (800 specks per image), most specks can be tracked for at most 5 s.

A detailed analysis was performed of collocation performance as a function of the ratio of the inter-speck distance, L_{IP}, to the characteristic distance that two randomly aligned specks must separate to be considered unbound, L_{ch} (given by Equation 1). The analysis includes a study of the influence of monitoring time, tₘ on collocation by comparing tₘ to the minimum evolution time, t_{ch} (given by Equation. 2). Recall that t_{ch} is the characteristic time it takes two randomly collocated particles to separate by L_{ch}. Briefly, at low particle densities (i.e., high L_{IP}/L_{ch}), random optical marker collocations are rare, and the bound fractions detected converge to the simulated values for all tₘ and \tilde{R}₁₂. For sufficiently high particle densities (L_{IP}/L_{ch} less than about 3.5), the data includes frequent random optical marker collocations, and bound optical markers (indicated by deterministically collocated specks) fractions for short observation times are over-predicted, as expected.

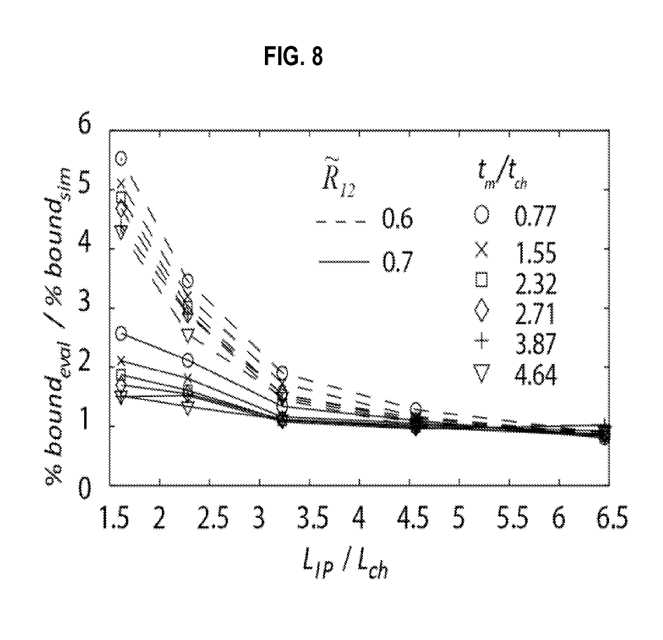

FIG. 8 is a graph 800 that illustrates example dependence of performance on various adjustable parameters, according to various embodiments. The horizontal axis 802 indicates speck density in terms of inter-speck distance L_{IP} divided by the distance between optical markers that is considered indicative of no binding to a target, L_{ch}, which is dimensionless. The vertical axis 804 indicates ratio of specks determined to represent optical markers bound to a target compared to the correct value, also dimensionless. For the data plotted, the correct value (denominator) is 3% and correct results are indicated by the value 1 on axis 804. The dashed lines indicate results for a maximum correlation threshold \tilde{R}₁₂ of 0.6; the solid lines for \tilde{R}₁₂ of 0.7. Different symbols represent results for different measurement times tₘ compared to t_{ch}, the characteristic time it takes two randomly collocated particles to separate. Open circle indicates a measurement time tₘ of 0.77 t_{ch}; a cross indicates a measurement time tₘ of 1.55 t_{ch}; an open square indicates a measurement time tₘ of 2032 t_{ch}; an open diamond indicates a measurement time tₘ of 2.71 t_{ch}; a plus indicates a measurement time tₘ of 3.87 t_{ch}; and an open triangle indicates a measurement time tₘ of 4.64 t_{ch}.

In all cases, increasing monitoring time increases certainty that collocation is deterministic and therefore indicative of optical markers bound to a target. For example, for a recommended value of \tilde{R}₁₂=0.7, and values of tₘ/t_{ch}=3.87 and L_{IP}/L_{ch}>3, the calculated values were within about 10% of the correct value. For example, for SNR=100 for the case of only 3% scatterers bound to targets, the method produced values between 2.7% and 3.3% in various simulations.

### Results of Bead Experiments

The experimental results for the solution containing two populations of 1 μm diameter, fluorescent beads and DNA targets of length 173 nucleotides (nt) are presented here. As described above, each bead type has a unique spectral signature and each is functionalized with unique probe molecules complimentary to the target (21 nt sequences for both Ch 1 and Ch 2 beads). Upon hybridization to the complimentary oligos, the target DNA bridges the two beads, creating a two-color bead doublet as depicted in FIG. 6A. The resulting bead solution containing three bead populations, red singlets, green singlets, and red-green bead hybrids, were loaded onto a microfluidic chip depicted in FIG. 6B and imaged with the dual-view system described above. A negative control experiment was also performed, using a bead suspension absent of DNA molecules of the DNA target type.

Monitoring collocation and other speck parameters in time improves the accuracy of estimates, as many sources of variation are uncorrelated in time. One example of this is the discretization error associated with imaging with a CCD array. For instance, speck intensities can vary significantly depending on the location of their center relative to the pixel edge. Another source of variation is associated with the random collocation of optical markers which can cause “spikes” in the correlation coefficient, R_{12,max}(t). Acquiring a single realization in time would therefore yield significant error in optical marker-to-target binding studies.

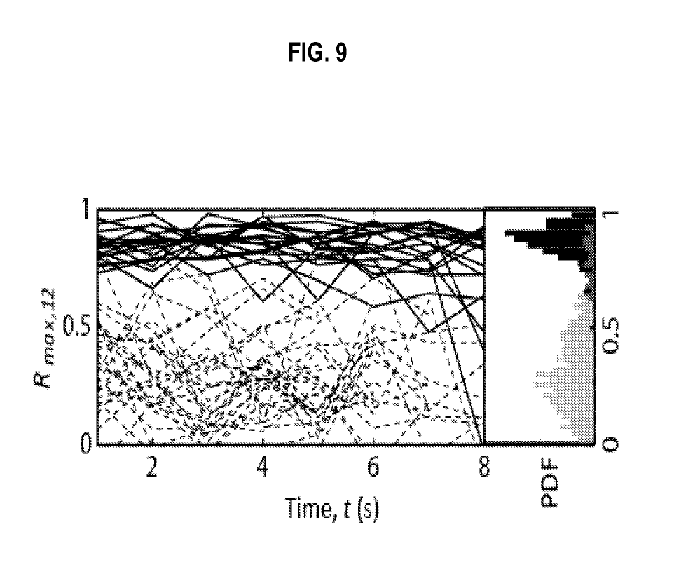

In FIG. 9, this phenomenon is shown by plotting the correlation coefficients evaluated from the experimentally obtained image sets. FIG. 9 is a graph 900 that indicates persistent correlation of specks in two spectral bands from the experiment of FIG. 6A through FIG. 6C, according to an embodiment. The first horizontal axis 902 indicates time in seconds; and, the vertical axis 904 indicates maximum collocation correlation, R_{12,max}, the collocation coefficient The second horizontal axis 903 indicate the probability density function of each value of R_{12,max}.

For the data depicted in FIG. 9, the minimum monitoring time is set to 8 s, which is approximately 3.2 times longer than the characteristic evolution time, t_{ch}, for this experiment. Filtering specks based on the 8 s of minimum monitoring time yielded time-resolved measurements of speck fluorescence, radius, and collocation for 163 red beads. The plot in the figure shows 50 representative traces of the time-resolved collocation correlation coefficient, R_{12,max}(t), for the red particles correlated with the green channel in the presence of DNA. The distribution of R_{12,max}(t) for all realizations of the 163 red beads is shown on the right hand side of FIG. 9. To accurately determine the fraction of bound beads (two-color doublets), the median collocation correlation threshold, \tilde{R}₁₂, was set to 0.65 and speck matches with intensity lower than 1 standard deviation from the mean of green speck matches filtered out. Then 20 traces of the 50 were judged by the embodiment of the method 450 as collocated specks from both channels, indicative of both beads bound to a DNA molecule of the DNA target type.

The black shaded region of the PDF is associated with the specks persistently collocated based on the beads being deterministically bound to the target; and, the grey shaded region is associated with the collocated specks that were not persistently collocated and thus due to one or more beads not deterministically bound to a DNA molecule of the target DNA type. The appropriateness of this collocation coefficient threshold at 0.65 is corroborated by the distinct difference between the bound (solid) and unbound (dashed) collocation coefficient traces. Each of the bound traces has a high mean (and high median, >0.8) value in time with a narrow variance, indicating high correlations associated with binding events. Meanwhile, the low mean (<0.3, and low median) value and large variance of unbound collocation traces indicate random, weakly correlated events, such as those associated with image noise or close proximity of a neighboring bead not bound to a target DNA of the DNA target type.

Integrated bead fluorescence were collected and analyzed to yield cytometry-like data, with beads as the optical marker types and DNA molecules of a certain type as the target type. Images of beads provided the specks of light from the optical markers. Individual beads in Ch 1 were identified and tracked and used in collocation correlation analysis with Ch 2 beads in contemporaneous images from a different time series. The algorithm was then run again, but starting by identifying and tracking beads in Ch 2 and collocating these with beads in Ch 1. This process helps corroborate collocation information and helps reduce the effect of bead images which may fail threshold tests associated with speck tracking but not collocation correlation analysis.

The fluorescent intensity of each bead was evaluated by integrating the intensity profile obtained from the non-linear Gaussian fitting routine. In the PMC-PC phase, the size-based threshold was set to eliminate beads with speck radii larger than the mean plus 1 times the standard deviation of the specks of the bead population in each image. The intensity-based threshold eliminated all features with intensity 1 times the standard deviation away from the mean of the bead population.

By performing analysis twice starting with alternate spectral channels, the intensity distribution of both Ch 1 and Ch 2 beads were obtained independently. These distributions were used to calibrate the intensity-based filters of the collocation correlation phase.

The collocation coefficient threshold, \tilde{R}₁₂, was set to 0.65 and the bead intensities and collocation correlations were monitored for 5 s, which is approximately 2 times t_{ch}. This embodiment of the method counted totals of 7903 (1010) red and 9351 (1292) green beads in the DNA-containing solution, and 7641 (1127) red and 9966 (1163) green beads for the negative control solution. In parenthesis is noted the number of beads that were tracked for 5 s or longer. In the solution containing DNA, the method 450 detected 418 red beads persistently collocated with green and 430 green beads persistently collocated with red.

In the negative control case, 9 red beads were persistently collocated with green, and 19 green beads persistently collocated with red. Visual inspection of the bead image field revealed that a small fraction of beads did form persistent two-color bead hybrids. It is hypothesized that this is a result of DNA contamination or non-specific bead-to-bead binding, or some combination.

The experiments and simulations performed here suggest that advantageous values for various parameters are as follows. Advantageously, SNR >5 and inter-particle distance of L_{IP}, >3 L_{ch}. For persistent collocation correlation, it is advantageous that tₘ>2 t_{ch}.

### 3. Computational Hardware Overview

FIG. 10 is a block diagram that illustrates a computer system 1000 upon which an embodiment of the invention may be implemented. Computer system 1000 includes a communication mechanism such as a bus 1010 for passing information between other internal and external components of the computer system 1000. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1000, or a portion thereof, constitutes a means for performing one or more steps of one or more methods described herein.

A sequence of binary digits constitutes digital data that is used to represent a number or code for a character. A bus 1010 includes many parallel conductors of information so that information is transferred quickly among devices coupled to the bus 1010. One or more processors 1002 for processing information are coupled with the bus 1010. A processor 1002 performs a set of operations on information. The set of operations include bringing information in from the bus 1010 and placing information on the bus 1010. The set of operations also typically include comparing two or more units of information, shifting positions of units of information, and combining two or more units of information, such as by addition or multiplication. A sequence of operations to be executed by the processor 1002 constitute computer instructions.

Computer system 1000 also includes a memory 1004 coupled to bus 1010. The memory 1004, such as a random access memory (RAM) or other dynamic storage device, stores information including computer instructions. Dynamic memory allows information stored therein to be changed by the computer system 1000. RAM allows a unit of information stored at a location called a memory address to be stored and retrieved independently of information at neighboring addresses. The memory 1004 is also used by the processor 1002 to store temporary values during execution of computer instructions. The computer system 1000 also includes a read only memory (ROM) 1006 or other static storage device coupled to the bus 1010 for storing static information, including instructions, that is not changed by the computer system 1000. Also coupled to bus 1010 is a non-volatile (persistent) storage device 1008, such as a magnetic disk or optical disk, for storing information, including instructions, that persists even when the computer system 1000 is turned off or otherwise loses power.

Information, including instructions, is provided to the bus 1010 for use by the processor from an external input device 1012, such as a keyboard containing alphanumeric keys operated by a human user, or a sensor. A sensor detects conditions in its vicinity and transforms those detections into signals compatible with the signals used to represent information in computer system 1000. Other external devices coupled to bus 1010, used primarily for interacting with humans, include a display device 1014, such as a cathode ray tube (CRT) or a liquid crystal display (LCD), for presenting images, and a pointing device 1016, such as a mouse or a trackball or cursor direction keys, for controlling a position of a small cursor image presented on the display 1014 and issuing commands associated with graphical elements presented on the display 1014.

In the illustrated embodiment, special purpose hardware, such as an application specific integrated circuit (IC) 1020, is coupled to bus 1010. The special purpose hardware is configured to perform operations not performed by processor 1002 quickly enough for special purposes. Examples of application specific ICs include graphics accelerator cards for generating images for display 1014, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1000 also includes one or more instances of a communications interface 1070 coupled to bus 1010. Communication interface 1070 provides a two-way communication coupling to a variety of external devices that operate with their own processors, such as printers, scanners and external disks. In general the coupling is with a network link 1078 that is connected to a local network 1080 to which a variety of external devices with their own processors are connected. For example, communication interface 1070 may be a parallel port or a serial port or a universal serial bus (USB) port on a personal computer. In some embodiments, communications interface 1070 is an integrated services digital network (ISDN) card or a digital subscriber line (DSL) card or a telephone modem that provides an information communication connection to a corresponding type of telephone line. In some embodiments, a communication interface 1070 is a cable modem that converts signals on bus 1010 into signals for a communication connection over a coaxial cable or into optical signals for a communication connection over a fiber optic cable. As another example, communications interface 1070 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1070 sends and receives electrical, acoustic or electromagnetic signals, including infrared and optical signals, that carry information streams, such as digital data.

The term computer-readable medium is used herein to refer to any medium that participates in providing information to processor 1002, including instructions for execution. Such a medium may take many forms, including, but not limited to, non-volatile media, volatile media and transmission media. Non-volatile media include, for example, optical or magnetic disks, such as storage device 1008. Volatile media include, for example, dynamic memory 1004. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1002, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1002, except for carrier waves and other signals.

Logic encoded in one or more tangible media includes one or both of processor instructions on a computer-readable storage media and special purpose hardware, such as ASIC *1020.

Network link 1078 typically provides information communication through one or more networks to other devices that use or process the information. For example, network link 1078 may provide a connection through local network 1080 to a host computer 1082 or to equipment 1084 operated by an Internet Service Provider (ISP). ISP equipment 1084 in turn provides data communication services through the public, world-wide packet-switching communication network of networks now commonly referred to as the Internet 1090. A computer called a server 1092 connected to the Internet provides a service in response to information received over the Internet. For example, server 1092 provides information representing video data for presentation at display 1014.

The invention is related to the use of computer system 1000 for implementing the techniques described herein. According to one embodiment of the invention, those techniques are performed by computer system 1000 in response to processor 1002 executing one or more sequences of one or more instructions contained in memory 1004. Such instructions, also called software and program code, may be read into memory 1004 from another computer-readable medium such as storage device 1008. Execution of the sequences of instructions contained in memory 1004 causes processor 1002 to perform the method steps described herein. In alternative embodiments, hardware, such as application specific integrated circuit 1020, may be used in place of or in combination with software to implement the invention. Thus, embodiments of the invention are not limited to any specific combination of hardware and software.

The signals transmitted over network link 1078 and other networks through communications interface 1070, carry information to and from computer system 1000. Computer system 1000 can send and receive information, including program code, through the networks 1080, 1090 among others, through network link 1078 and communications interface 1070. In an example using the Internet 1090, a server 1092 transmits program code for a particular application, requested by a message sent from computer 1000, through Internet 1090, ISP equipment 1084, local network 1080 and communications interface 1070. The received code may be executed by processor 1002 as it is received, or may be stored in storage device 1008 or other non-volatile storage for later execution, or both. In this manner, computer system 1000 may obtain application program code in the form of a signal on a carrier wave.

Various forms of computer readable media may be involved in carrying one or more sequence of instructions or data or both to processor 1002 for execution. For example, instructions and data may initially be carried on a magnetic disk of a remote computer such as host 1082. The remote computer loads the instructions and data into its dynamic memory and sends the instructions and data over a telephone line using a modem. A modem local to the computer system 1000 receives the instructions and data on a telephone line and uses an infrared transmitter to convert the instructions and data to a signal on an infrared carrier wave serving as the network link 1078. An infrared detector serving as communications interface 1070 receives the instructions and data carried in the infrared signal and places information representing the instructions and data onto bus 1010. Bus 1010 carries the information to memory 1004 from which processor 1002 retrieves and executes the instructions using some of the data sent with the instructions. The instructions and data received in memory 1004 may optionally be stored on storage device 1008, either before or after execution by the processor 1002.

FIG. 11 illustrates a chip set 1100 upon which an embodiment of the invention may be implemented. Chip set 1100 is programmed to perform one or more steps of a method described herein and includes, for instance, the processor and memory components described with respect to FIG. 10 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1100, or a portion thereof, constitutes a means for performing one or more steps of a method described herein.

In one embodiment, the chip set 1100 includes a communication mechanism such as a bus 1101 for passing information among the components of the chip set 1100. A processor 1103 has connectivity to the bus 1101 to execute instructions and process information stored in, for example, a memory 1105. The processor 1103 may include one or more processing cores with each core configured to perform independently. A multi-core processor enables multiprocessing within a single physical package. Examples of a multi-core processor include two, four, eight, or greater numbers of processing cores. Alternatively or in addition, the processor 1103 may include one or more microprocessors configured in tandem via the bus 1101 to enable independent execution of instructions, pipelining, and multithreading. The processor 1103 may also be accompanied with one or more specialized components to perform certain processing functions and tasks such as one or more digital signal processors (DSP) 1107, or one or more application-specific integrated circuits (ASIC) 1109. A DSP 1107 typically is configured to process real-world signals (e.g., sound) in real time independently of the processor 1103. Similarly, an ASIC 1109 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1103 and accompanying components have connectivity to the memory 1105 via the bus 1101. The memory 1105 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1105 also stores the data associated with or generated by the execution of one or more steps of the methods described herein.

### 4. Nucleotide Sequences

In alternative embodiments, one or more molecules include one or more of the nucleotide sequences described next.

Deoxyribonucleic acid (DNA) is a usually double-stranded long molecule that encodes other shorter molecules, such as proteins, used to build and control all living organisms. DNA is composed of repeating chemical units known as “nucleotides” or “bases.” There are four bases: adenine, thymine, cytosine, and guanine, represented by the letters A, T, C and G, respectively. Adenine on one strand of DNA always binds to thymine on the other strand of DNA, and guanine on one strand always binds to cytosine on the other strand; such bonds are called base pairs. Any order of A, T, C and G is allowed on one strand, and that order determines the complementary order on the other strand. The actual order determines the function of that portion of the DNA molecule. Information on a portion of one strand of DNA can be captured by ribonucleic acid (RNA) that also comprises a chain of nucleotides in which uracil (U) replaces thymine (T). Determining the order, or sequence, of bases on one strand of DNA or RNA is called sequencing. A portion of length k bases of a strand is called a k-mer; and specific short k-mers are called oligonucleotides or oligomers or “oligos” for short.

It is known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” is known in the art to refer to the region between the translation initiation codon and the translation termination codon. It is also known in the art that variants can be produced through the use of alternative signals to start or stop transcription and that pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

In the context of various embodiments, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides that can hydrogen bond with each other. Thus, “specifically hybridizable” and “complementary” are terms that are used to indicate a sufficient degree of complementarity or precise pairing such that stable and specific binding occurs between the nucleic acid and the DNA or RNA target.

Various conditions of stringency can be used for hybridization as is described below. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 631-636 Aqueous and nonaqueous methods are described in that reference and either can be used. Specific hybridization conditions referred to herein are as follows: (1) low stringency hybridization conditions in 6 times sodium chloride/sodium citrate (SSC) at about 45° C., followed by two washes in 0.2 times SSC, 0.1% SDS at least at 50° C. (the temperature of the washes can be increased to 55° C. for low stringency conditions); (2) medium stringency hybridization conditions in 6 times SSC at about 45° C., followed by one or more washes in 0.2 times SSC, 0.1% SDS at 60° C.; (3) high stringency hybridization conditions in 6 times SSC at about 45° C., followed by one or more washes in 0.2 times SSC, 0.1% SDS at 65° C.; and preferably (4) very high stringency hybridization conditions are 0.5M sodium phosphate, 7% SDS at 65° C., followed by one or more washes at 0.2 times SSC, 1% SDS at 65° C. Very high stringency conditions (4) are the preferred conditions and the ones that should be used unless otherwise specified.

Nucleic acids in the context of various embodiments include “oligonucleotides,” which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. DNA/RNA chimeras are also included.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′,3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Oligonucleotides containing modified backbones or non-natural internucleoside linkages can be used. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In some oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

Some embodiments of some embodiments use oligonucleotides with phosphorothioate backbones and oligonucleosides with heteroatom backbones, and in particular —CH₂—NH—O—CH₂—, —CH₂—N(CH₃)—O—CH₂— [known as a methylene(methylimino) or MMI backbone], —CH₂—O—N(CH₃)—CH₂—, —CH₂—N(CH₃)—N(CH₃)—CH₂— and —O—N(CH₃)—CH₂—CH₂—[wherein the native phosphodiester backbone is represented as —O—P—O—CH₂] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)—OCH₃, O(CH₂).sub.nNH₂, O(CH₂)ₙCH₃, O(CH₂)ₙONH₂, and O(CH₂)ₙON[(CH₂).sub.nCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′-O—CH₂CH₂OCH₃, also known as 2′-O-(2-methoxyethyl) or 2′-MOE) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylamino-ethoxyethyl or 2′-DMAEOE), i.e., 2′-O—CH₂—O—CH₂—N(CH₂)₂, also described in examples hereinbelow.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy(2′-O—CH₃), 2′-aminopropoxy (2′-OCH₂CH₂CH₂NH₂), 2′-allyl (2′-CH₂—CH═CH₂), 2′-O-allyl (2′-O-CH₂—CH═CH₂) and 2′-fluoro(2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine. (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C.ident.C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808; those disclosed in The Concise Encyclopedia of Polymer Science and Engineering, pages 858-859, ed. J. I. Kroschwitz (John Wiley & Sons, 1990); those disclosed by Englisch et al., Angewandte Chemie, International Ed., 1991, 30, 613; and those disclosed by Y. S. Sanghvi, Antisense Research and Applications, pp. 289-302 (eds. S. T. Crooke and B. Lebleu, CRC Press, 1993). Certain of these nucleobases are particularly useful for increasing the binding affinity of the oligomeric compounds of some embodiments. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941.

Another modification of the oligonucleotides for use in some embodiments involves chemically linking to the oligonucleotide one or more moieties or conjugates which enhance the activity, cellular distribution or cellular uptake of the oligonucleotide. The compounds of some embodiments can include conjugate groups covalently bound to functional groups such as primary or secondary hydroxyl groups. Conjugate groups of some embodiments include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of various embodiments, include groups that improve oligomer uptake, enhance oligomer resistance to degradation, and/or strengthen sequence-specific hybridization with RNA. Groups that enhance the pharmacokinetic properties, in the context of various embodiments, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety, cholic acid, a thioether (e.g., hexyl-S-tritylthiol), a thiocholesterol, an aliphatic chain (e.g., dodecandiol or undecyl residues), a phospholipid (e.g., di hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety. Oligonucleotides of some embodiments may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941.

It is not necessary for all positions in a given compound to be uniformly modified, and in fact more than one of the aforementioned modifications may be incorporated in a single compound or even at a single nucleoside within an oligonucleotide. “Chimeric” compounds or “chimeras,” in the context of various embodiments, are oligonucleotides that contain two or more chemically distinct regions, each made up of at least one monomer unit, i.e., a nucleotide, in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

The oligonucleotides used in accordance with various embodiments may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

### 5. Extensions, Alternatives and Modification

In the foregoing specification, descriptions have been provided with reference to specific embodiments. It will, however, be evident that various modifications and changes may be made thereto without departing from the broader spirit and scope of the attached claims. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### REFERENCES

Adrian, R. J. and Westerweel, J. Particle image velocimetry, vol. 30. Cambridge University Press: 2010.
Brown, M. and Wittwer, C. Clinical Chemistry. 2000, 46 (8), 1221-1229.
Chandrasekhar, S. Reviews of modern physics. 1943, 15 (1), 1.
Crooke, S. T., Graham, M. J., Zuckerman, J. E, Brooks, D., Conklin, B. S., Cummins, L. L., Greig, M. J., Guinosso, C. J., Kornbrust, D., Manoharan, M., Sasmor, H. M., Schleich, T., Tivel, K. L., Griffey, R. H. Journal of Pharmacology and Experimental Therapeutics 1996, 277, 923-937.
Darzynkiewicz, Z.; Bedner, E.; Li, X.; Gorczyca, W. and Melamed, M. R. Experimental cell research. 1999, 249 (1), 1-12.
De Rosa, S. C.; Brenchley, J. M. and Roederer, M. Nature Medicine. 2003, 9 (1), 112-117.
Dunbar, S. A. Clinica Chimica Acta. 2006, 363 (1), 71-82.
Einstein, A. Annalen der Physik. 1905, 17 (549-560), 16.
Inagaki, T.; Arakawa, E.; Hamm, R. and Williams, M. Physical Review B. 1977, 15 (6), 3243.
Integrated DNA Technologies, “Strategies for Attaching Oligonucleotides to Solid Supports,” publisher: Integrated DNA Technologies, Inc., Coralville, Iowa, 19 pp, 2010.
Inoue, S. and Spring, K. R. Video Microscopy: The Fundamentals (The Language of Science). Plenum, New York: 1997.
Kabanov et al. FEBS Letters. 1990, 259, 327-330.
Kamentsky, L. A. and Kamentsky, L. D. Cytometry. 1991, 12 (5), 381-387.
Keane, R. D. and Adrian, R. J. Measurement Science and Technology. 1990, 1 (11), 1202.
Keane, R.; Adrian, R. and Zhang, Y. Measurement Science and Technology. 1995, 6 (6), 754.
Kinosita, K.; Itoh, H.; Ishiwata, S.; Hirano, K.; Nishizaka, T. and Hayakawa, T. The Journal of Cell Biology. 1991, 115 (1), 67-73.
Lenz, D.; Gerstner, A. O.; Laffers, W.; Steinbrecher, M.; Bootz, F. and Tarnok, A. Proc. of Biomedical Optics 2003, pp 364-374.
Leslie, D. C., et al. Journal of the American Chemical Society. 2012, 134 (12), 5689-5696.
Letsinger, R. L., Zhang, G., Sun, D. K., Ikeuchi, T.; and Sarin, P. S. Proc. Natl. Acad. Sci. USA. 1989, 86, 6553-6556.
Manoharan, M., Johnson, L. K., McGee, D. P. C., Guinosso, C. J., Ramasamy, K., Springer, R. H., Bennett, C. F., Ecker, D. J., Vickers, T., Cowsert, L., and Cook, P. D. Ann. N.Y. Acad. Sci. 1992, 660, 306-309.
Manoharan, M., Johnson, L. K., Tivel, K. L, Springer, R. H., and Cook, P. D. Bioorganic and Medicinal Chemistry Letters 1993, 3, 2765-2770.
Manoharan, M., Johnson, L. K., Bennett, C. F., Vickers, T. A., Ecker, D. J., Cowsert, D. M., Freier, S. M., Cook, P. D. Bioorganic and Medicinal Chemistry Letters. 1994, 4 (8), 1053-1060.
Manoharan, M., Tivel, K. L., Andrade, L. K., Mohan, V., Condon, T. P., Bennett, C. F., and Cook, P. D. Nucleosides & Nucleotides. 1995, 14 (3-5), 969-973.
Manoharan, M., Tivel, K. L., Andrade, L. K., Cook, P. D. Tetrahedron Letters. 1995, 36, 3651-3654.
Martin, P. Helvetica Chimica Acta. 1995, 78, 486-504.
Meinhart, C.; Wereley, S. and Gray, M. Measurement Science and Technology. 2000, 11 (6), 809.
Mishra, R. K., Moreau, C., Ramazeilles, C., moreau, S., Bonnet, J., Toulme, J.-J. Biochimica et Biophysica Acta: Gene Structure and Expression. 1995, 1264, 229-237.
Nam, J.-M.; Stoeva, S. I. and Mirkin, C. A. Journal of the American Chemical Society. 2004, 126 (19), 5932-5933.
Oberhauser, B., and Wagner, E. Nucleic Acids Research. 1992, 20 (3), 533-538.
Ortiz, M. E. and Endy, D. Journal of biological engineering. 2012, 6 (1), 1-12.
Perfetto, S. P.; Chattopadhyay, P. K. and Roederer, M. Nature Reviews Immunology. 2004, 4 (8), 648-655.
Quirke, P. and Dyson, J. The Journal of pathology. 1986, 149 (2), 79-87.
Raffel, M.; Willert, C. E.; Wereley, S. T. and Kompenhans, J. Particle Image Velocimetry—A Pratical Guide. 2 ed.; Springer Press: Heidelberg, 2007.
Saison-Behmoaras, T., Tocque, B., Rey, I., Chassignol, M., Thuong, N. T., Helene, C. EMBO J. 1991, 10 (5), 1111-1118.
Sanghvi, Y. S., Crooke, S. T. and Lebleu, B., eds. Antisense Research and Applications. 1993, CRC Press, Boca Raton, pp. 276-278.
Shapiro, H. M. Practical flow cytometry. 4 ed.; Wiley-Liss: NY, N.Y., 2003.
Shapiro, H. M. and Perlmutter, N. G. Cytometry Part B: Clinical Cytometry. 2008, 74 (51), S152-S164.
Shea, R., Marster, J. C., and Bischofberger, N. Nucleic Acids Research. 1990, 18(13), 3777-3783.
Svinarchuk, F. P., Konevetz, D. A., Pliasunova, O. A., Pokrovsky, A. G., and Vlassov, V. V. Biochimie. 1993, 75(1-2), 49-54.
Takehara, K.; Adrian, R. J.; Etoh, G. T. and Christensen, K. T. Experiments in Fluids. 2000, 29 (1), S034-S041.
Tárnok, A. and Gerstner, A. O. Cytometry. 2002, 50 (3), 133-143.
Westerweel, J. Measurement Science and Technology. 1997, 8 (12), 1379.
Wilson, R.; Cossins, A. R. and Spiller, D. G. Angewandte Chemie International Edition. 2006, 45 (37), 6104-6117.
Zou, Y.; Heinemann, F. M.; Grosse-Wilde, H.; Sireci, G.; Wang, Z.; Lavingia, B. and Stastny, P. Human immunology. 2006, 67 (3), 230-237.

1. A method comprising:
providing a supply of at least two optical marker types that scatter or emit light under a corresponding number of different conditions, wherein the at least two optical marker types are configured to collocate with a single target type;
introducing a fluid that includes the supply into an interrogation region of a channel;
serially imaging the interrogation region under the corresponding number of different conditions to produce a plurality of time series of images wherein each time series of images detects light from a single optical marker type of the at least two optical marker types;
determining, in a first time series of the plurality of time series of images, a path of a moving speck of light from an individual optical marker of a first optical marker type of the at least two optical marker types based on particle tracking velocimetry and rate of any fluid flow of the fluid; and
determining whether the path corresponds to the target type based on persistence of collocation of the speck of light from the individual optical marker of the first optical marker type with a moving speck of light from each of other optical marker types of the at least two optical marker types, wherein the other optical marker types are different from the first optical marker type.

2. A method as recited in claim 1, wherein the speck of light from each of other optical marker types is determined to collocate with the speck of light from the individual optical marker of the first optical marker type if the specks are located within a collocation distance that is based on size of the target type and size of the at least two optical marker types.

3. (canceled)

4. A method as recited in claim 1, further comprising determining a characteristic of the fluid based on whether the path corresponds to the target type, wherein the characteristic is selected from a group comprising: presence of the target type, amount of the target type, relative amounts of multiple different target types, diagnosis of condition of subject which contributed a component of the fluid, and effectiveness of treatment given to the subject.

5. A method as recited in claim 1, the method further comprising:
determining, in the time series of images, a plurality of paths of corresponding specks of light from corresponding individual optical markers of the first optical marker type; and,
determining a number of the plurality of paths that correspond to the target type based on persistence of collocation of a speck of light from each of the other optical marker types for each path of the plurality of paths.

6.-7. (canceled)

8. A method as recited in claim 1, wherein:
the target type is a cell type that expresses a first particular molecule and a second particular molecule; and
the cell type has been genetically engineered so that a first fluorescent protein that serves as a first optical marker type of the at least two optical marker types is expressed when the first particular molecule is expressed.

9.-10. (canceled)

11. A method as recited in claim 1, wherein:
the target type is a cell type that expresses a first particular molecule and a second particular molecule;
the first particular molecule is a receptor on a membrane of the cell type; and
a first fluorescent antibody that serves as a first optical marker type of the at least two optical marker types binds to the receptor.

12. (canceled)

13. A method as recited in claim 1, wherein:
the target type is a molecule with a particular unique sequence of hybridizing sites;
a first fluorescent labeled probe that binds to a first portion of the particular unique sequence serves as a first optical marker type of the at least two optical marker types; and
a second fluorescent labeled probe that binds to a different second portion of the particular unique sequence serves as a different second optical marker type of the at least two optical marker types.

14. (canceled)

15. A method as recited in claim 1, wherein:
providing the supply further comprises providing a supply of a multiplexed optical marker type that scatters or emits light under a corresponding different multiplexed condition and is configured to collocate with at least one other optical marker type at a single different target type;
serially imaging the interrogation region further comprises serially imaging the interrogation region under conditions that cause the multiplexed optical marker to emit light, to produce a multiplexed time series of images that detects light from the multiplexed optical marker type;
the method further comprises,
determining, in the multiplexed time series of images, a path of a speck of light from the multiplexed optical marker type; and
determining whether the path corresponds to the multiplexed target type based on persistence of collocation of a speck of light from the at least one other optical marker type.

16. (canceled)

17. A method as recited in claim 1, wherein persistence of collocation of the speck of light from each of the other optical marker types is based on persistence of a correlation measure above a correlation threshold.

18. A method as recited in claim 17, wherein the correlation measure is a maximum correlation among a plurality of correlations within a collocation area between corresponding portions of contemporaneous images of two of the plurality of time series.

19. A non-transitory computer-readable medium carrying one or more sequences of instructions, wherein execution of the one or more sequences of instructions by one or more processors causes an apparatus to perform at least the following:
obtaining a plurality of time series of images, each image representing light measured in an interrogation area of a fluid under conditions that cause one optical marker type of at least two optical marker types to emit or scatter light, wherein
each different time series of images indicates light measured from a different single optical marker type of the at least two optical marker types, and
the at least two optical marker types are configured to collocate with a single target type;
determining on a processor, in the time series of images, a path of a moving speck of light from an individual optical marker of a first optical marker type of the at least two optical marker types based on particle tracking velocimetry and rate of any fluid flow of the fluid;
determining on a processor whether the path corresponds to the target type based on persistence of collocation of the speck of light from the individual optical marker of the first optical marker type with a moving speck of light from each of other optical marker types of the at least two optical marker types, wherein the other optical marker types are different from the first optical marker type; and
presenting, on a display device, output data based on the persistence of collocation.

20. A non-transitory computer-readable medium as recited in claim 19, wherein persistence of collocation of the speck of light from each of the other optical marker types is based on persistence of a correlation measure above a correlation threshold.

21. A non-transitory computer-readable medium as recited in claim 20, wherein the correlation measure is a maximum correlation among a plurality of correlations within a collocation area between corresponding portions of contemporaneous images of two of the plurality of time series.

22. A non-transitory computer-readable medium as recited in claim 21, wherein the collocation area is based on an expected size for the target type and values for zero or more parameters selected from a group comprising:
expected sizes of the at least two optical marker types;
expected positions of the at least two optical marker types on the target type; and
expected number of pixels over which is spread light from the at least two optical marker types.

23. A non-transitory computer-readable medium as recited in claim 21, wherein each correlation of the plurality of correlations is computed with a different shift of pixels in each of one or two dimensions between the two contemporaneous images.

24. A non-transitory computer-readable medium as recited in claim 20, wherein determining whether the path corresponds to the target type based on persistence further comprises computing a persistence time during which consecutive images in the time series produced the correlation measure above the correlation threshold.

25. A non-transitory computer-readable medium as recited in claim 24, wherein determining whether the path corresponds to the target type based on persistence further comprises determining whether the persistence time is greater than a value of a time threshold parameter.

26. (canceled)

27. A non-transitory computer-readable medium as recited in claim 19, wherein determining the path of the speck of light from the individual optical marker further comprises:
determining a local group velocity of a second optical marker type of the at least two optical marker types based on a local shift that leads to a maximum correlation between consecutive images from the time series of images under conditions that causes the second optical marker type to emit or scatter light; and
determining a next position along the path based on a position for the speck of light from the individual optical marker in a previous image of the time series, the local group velocity, and a next image of the time series.

28. A non-transitory computer-readable medium as recited in claim 19, wherein the apparatus is further configured to perform the steps of
obtaining a multiplexed time series of images, each image representing optical emissions in the interrogation area of fluid under different multiplexed conditions that cause only a different multiplexed optical marker to emit or scatter light, wherein the multiplexed optical marker and at least one other optical marker are configured to collocate with a different single target type;
determining, in the time series of images, a multiplexed path of a speck of light from the multiplexed optical marker; and
determining whether the multiplexed path corresponds to the multiplexed target type based on persistence of collocation of a speck of light from the at least one other optical marker type.

29.-30. (canceled)

31. A kit comprising:
a supply, for each target type, of at least two optical marker types that scatter or emit light under a corresponding number of different conditions and are all configured to collocate only with the target type; and
a computer-readable medium carrying one or more sequences of instructions, wherein execution of the one or more sequences of instructions by one or more processors causes the one or more processor to perform at least the following:
obtain a plurality of time series of images, each image representing light measured in an interrogation area of a fluid under conditions that cause one optical marker type of at least two optical marker types to emit or scatter light, wherein
each different time series of images indicates light measured from a different single optical marker type of the at least two optical marker types, and
the at least two optical marker types are configured to collocate with a single target type;
determine, in the time series of images, a path of a moving speck of light from an individual optical marker of a first optical marker type of the at least two optical marker types based on particle tracking velocimetry and rate of any fluid flow of the fluid; and
determine whether the path corresponds to the target type based on persistence of collocation of the speck of light from the individual optical marker of the first optical marker type with a moving speck of light from each of other optical marker types of the at least two optical marker types, wherein the other optical marker types are different from the first optical marker type.

32. (canceled)


SEQUENCE LISTING (3 sequences)

SEQ ID NO: 1 - DNA, 21 bp, Artificial Sequence
  Synthetic oligonucleotide
cacaaagtggtaagcgccctc

SEQ ID NO: 2 - DNA, 21 bp, Artificial Sequence
  Synthetic oligonucleotide
cggattggagtctgcaactcg

SEQ ID NO: 3 - DNA, 75 bp, Artificial Sequence
  Synthetic oligonucleotide
aaacgagttgcagactccaatccgaaaagaagtaggtagcttaaccttcgggagggcgcttaccactttgtgttt